(19) United States
(12) Patent Application Publication
(10) Pub. No.: US 2024/0415985 A1
(43) Pub. Date: Dec. 19, 2024

(54) PHOTOCLEAVABLE PRODRUG-BASED NANOMEDICINE FOR IN-SITU MONITORABLE CANCER THERAPY

(71) Applicant: The University of Hong Kong, Hong Kong (CN)

(72) Inventors: Weiping WANG, Hong Kong (CN); Kaiqi LONG, Hong Kong (CN); Yifan WANG, Hong Kong (CN)

(21) Appl. No.: 18/702,650

(22) PCT Filed: Oct. 11, 2022
(86) PCT No.: PCT/CN2022/124524

§ 371 Date: Apr. 18, 2024

Related U.S. Application Data
(60) Provisional application No. 63/262,789, filed on Oct. 20, 2021.

(51) Int. Cl. A61K 49/00

(52) U.S. Cl. CPC A61K 49/0052; A61K 49/0032

(57) ABSTRACT

The subject invention pertains to photocleavable prodrugs that facilitate the controllable drug delivery to the target sites modulated by light irradiation, including a photocleavable boron-dipyrromethene-derived prodrug and a dye, which achieved both high prodrug loading capacity (˜99%) and efficient light-triggered prodrug activation. The incorporation of the dye not only stabilized the nanoparticles and contributed tumor targeting as usual. but also exhibited degradation after light irradiation and in-situ monitoring of nanoparticle dissociation by fluorescent imaging.

### BACKGROUND OF THE INVENTION

Prodrug strategy provides vast possibilities for improving the efficacy of chemotherapeutic drugs by conjugating them with functional moieties [1, 2]. So far, prodrugs that respond to various stimuli, including pH [3-5], enzyme [6, 7], ultrasound [8], heat [9, 10] and light [11, 12], have been developed to reduce the drug toxicity to normal tissues while retained the activity at diseased lesions. Among all the stimuli, light is one of the most convenient and effective triggers, providing precise spatiotemporal control with low expense. [13]. Recently, some photoresponsive prodrugs have been developed with tailor-made structures composed of photocleavable moieties such as coumarin and boron-dipyrromethene (BODIPY) for photoactivable chemotherapy [14-16]. After systemically administrating the photoresponsive prodrug, light irradiation can be applied locally at the diseased site, thereby realizing light-controlled drug release. For instance, Lv et al. previously developed a BODIPY-chlorambucil prodrug (BC) of which the BODIPY group can be efficiently cleaved upon light irradiation, thus achieved effective antitumor effect with the utilization of an LED lamp [17]. However, due to the hydrophobicity of photocleavable groups, water solubility of most photoresponsive prodrugs is poor, which hindered their utility in vivo [18, 19].

Light-responsive drug delivery systems have been shown to be useful for cancer treatment by applying light to trigger drug release and local accumulation. The wavelength of light used for photoresponsive drug delivery is of great importance [11]. The most frequently used light for the activation of the systems is UV (<400 nm) light, of which the tissue penetration is limited [20]. The longer-wavelength light would allow deeper tissue penetration [21]. Green light has been reported to trigger the phototargeting of nanoparticles in a subcutaneous tumor model and trigger the drug release from nanoparticles in the eye [23]. For diseased lesions deep in the body, light delivery by optical fibers may be an option [24, 25].

So far, there remains a need for photoresponsive prodrug delivery systems with high loading capacity, efficient response process and sufficient stability.

### BRIEF SUMMARY OF THE INVENTION

The subject invention pertains to a photoresponsive nanomedicine composition and methods of using said composition by fabricating multifunctional photoresponsive nanomedicine by co-assembling a boron-dipyrromethene-derived compound (BODIPY-derived compound) and a dye. The boron-dipyrromethene-derived compound serving as prodrug comprises one or more selected from boron-dipyrromethene-chlorambucil (BC), boron-dipyrromethene-naproxen (BN), boron-dipyrromethene-benzyloxycinnamic acid (BBA), and boron-dipyrromethene-dopamine (BD). The dye is a near-infrared dye, including near-infrared cyanine dyes, e.g. IR783, IR820, and indocyanine green (ICG). IR783, a water-soluble dye, is commercially available and used for in vivo imaging [26]. In certain embodiments, the dye, such as, for example, IR783, IR820 and/or ICG, co-assembled in the photoresponsive prodrug-based nanoparticles, can provide versatile functions by serving as: 1) stabilizer that can be degraded after light irradiation; 2) targeting moiety with sulfate groups that enhance the caveolin-1 (CAV-1) mediated transcytosis in tumors; 3) fluorescent imaging agent for in-situ monitoring of nanoparticle disassembly.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIGS. 1A-1J. Preparation and characterization of IR783/BC NPs and other prodrug/dye NPs. (FIG. 1A) Nanoparticle preparation for IR783/BC NPs by the flash precipitation method. (FIG. 1B) Representative images of free IR783, free BC prodrug, and IR783/BC NPs in aqueous solution. (FIG. 1C) Size and PDI change of IR783/BC NPs when prepared with different concentrations of IR783 as the assemble matrix (n=3). (FIGS. 1D-1E) Representative size and zeta potential distribution of IR783/BC NPs. (FIG. 1F) Stability test of IR783/BC NPs at 37° C. in PBS solution for 48 h. (FIG. 1G) Nanoparticle preparation for prodrug/dye NPs by the flash precipitation method, wherein the prodrug is BODIPY-derived compound, including BC, BN, BBA and/or BD; the dye is near-infrared cyanine dye, including IR783, IR820, and/or ICG. (FIG. 1H) NMR spectra of BC, BN, BBA and BD. (FIG. 11) Absorption spectra of IR783, IR820 and ICG. (FIG. 1J) Size distribution of prodrug/dye NPs other than IR783/BC NPs.

FIGS. 2A-2M. Light-triggered photocleavage, NPs dissociation and drug release. (FIG. 2A) TEM image of IR783/BC NPs without light irradiation. (FIG. 2B) TEM image of IR783/BC

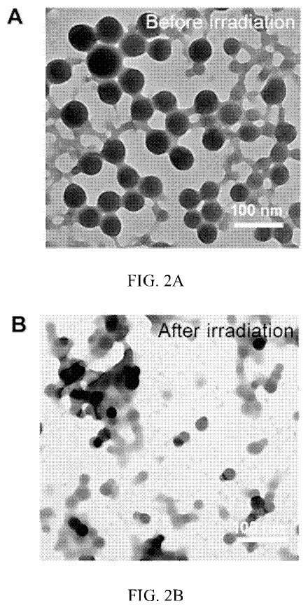

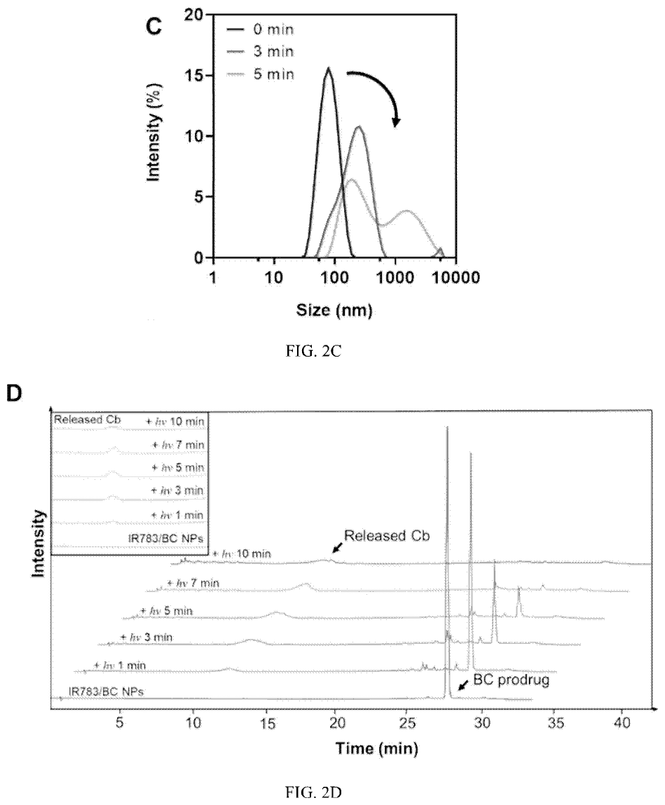

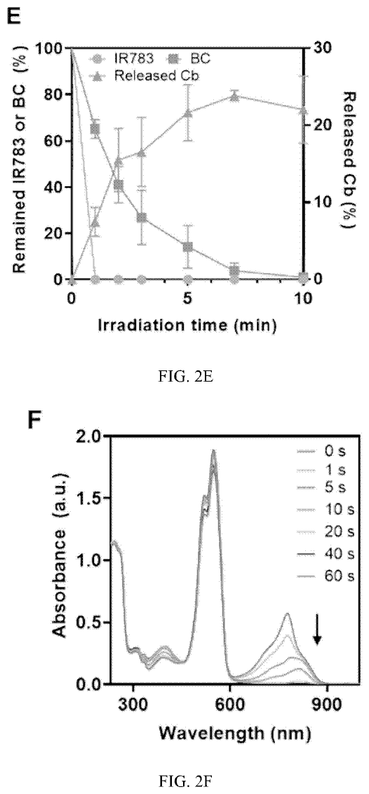

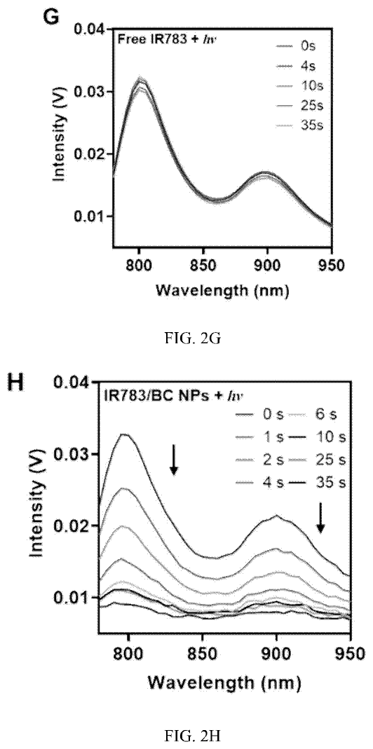

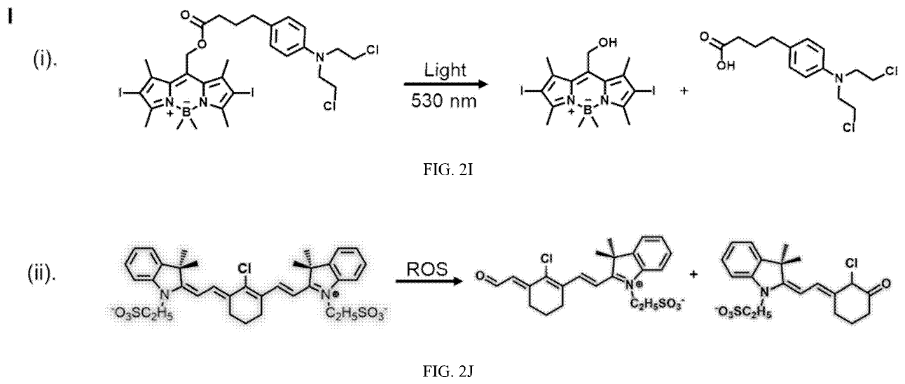

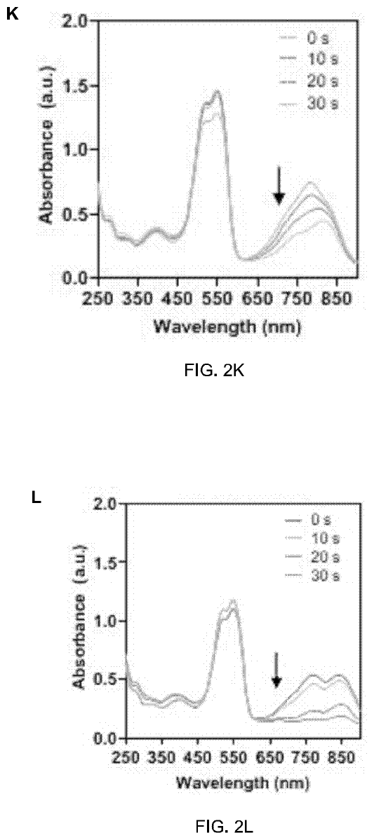

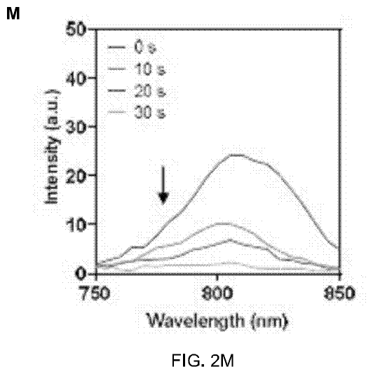

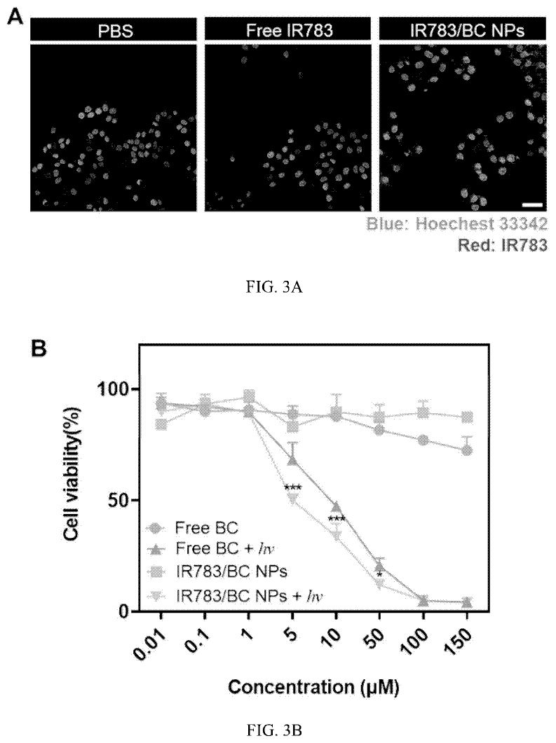

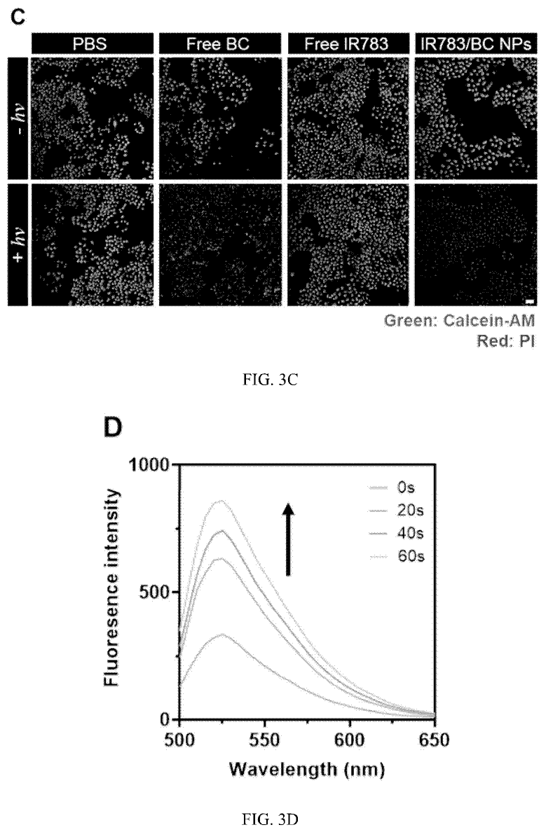

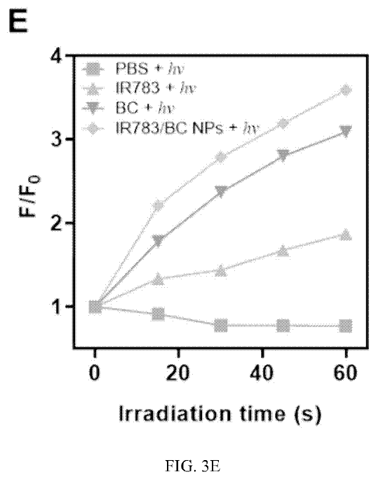

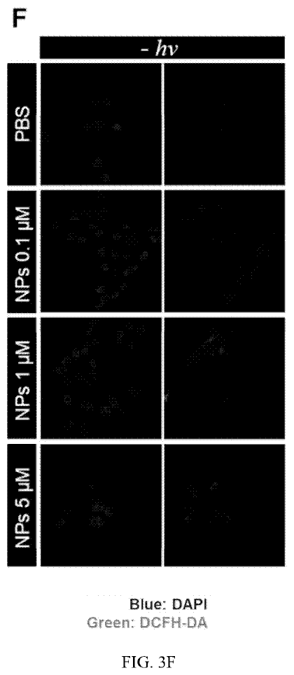

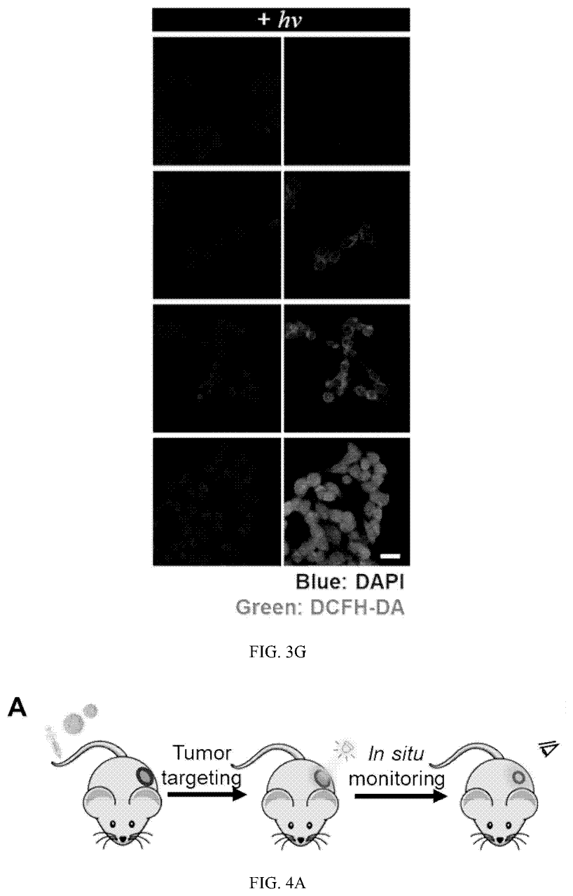

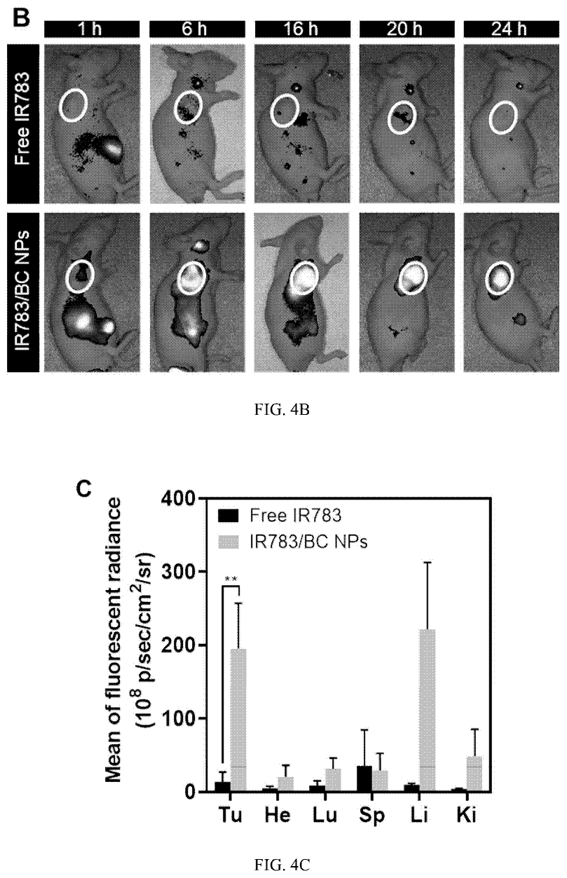

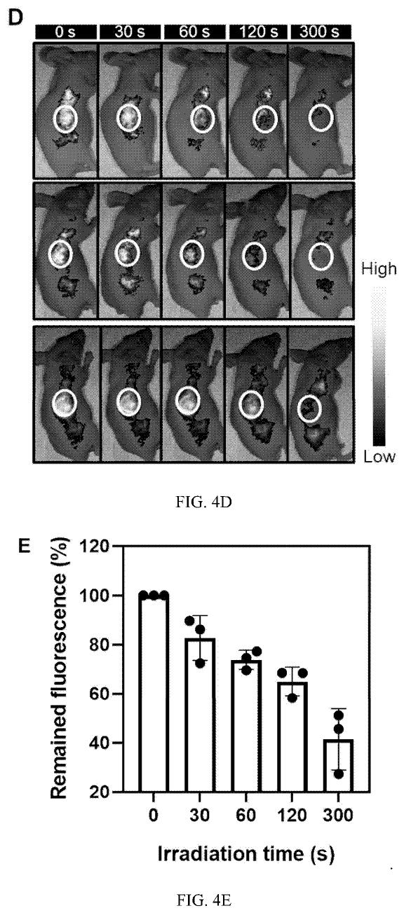

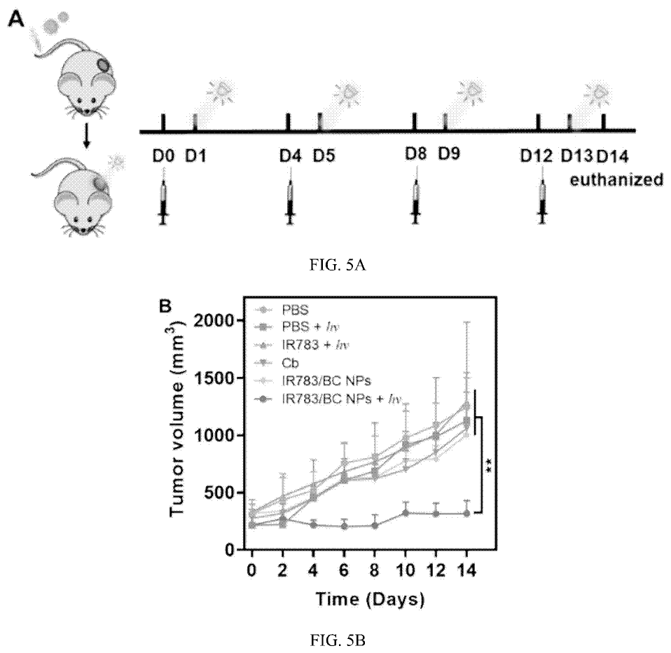

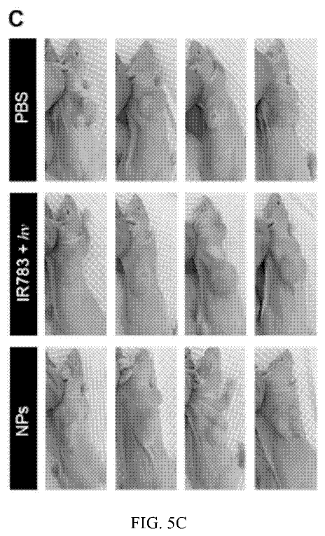

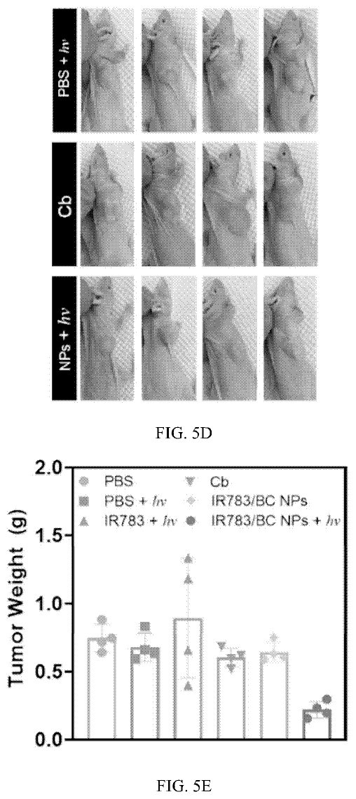

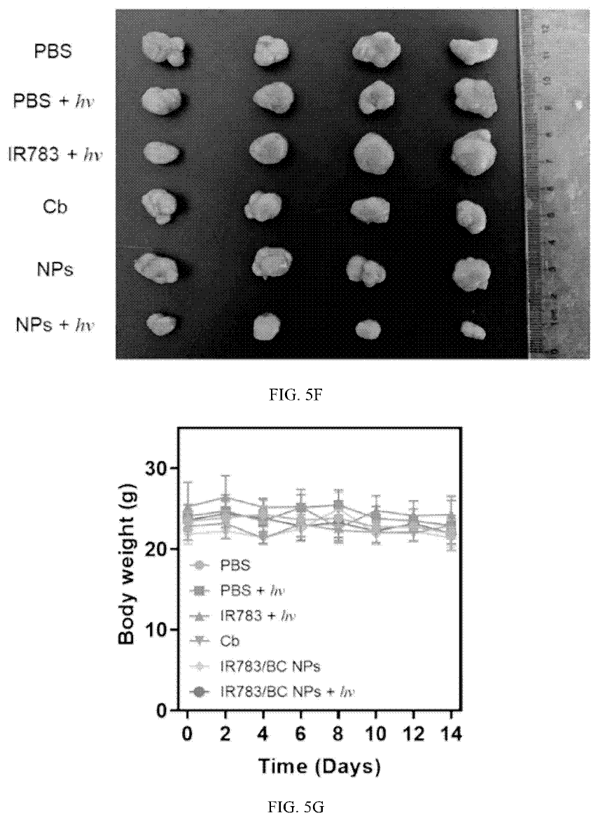

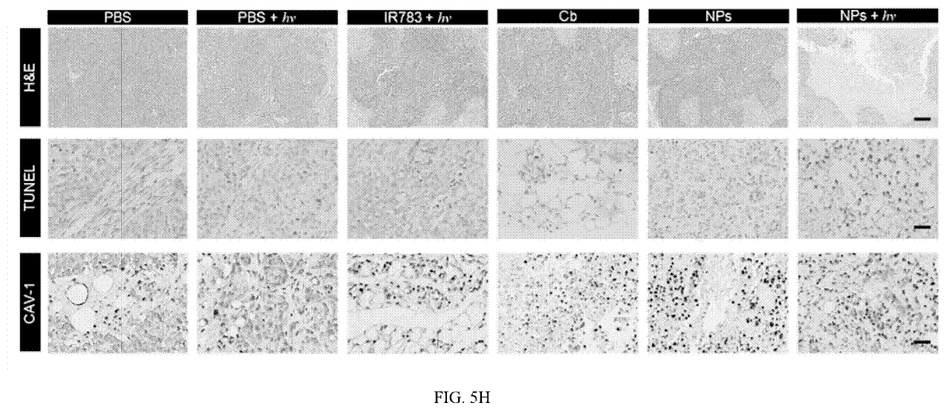

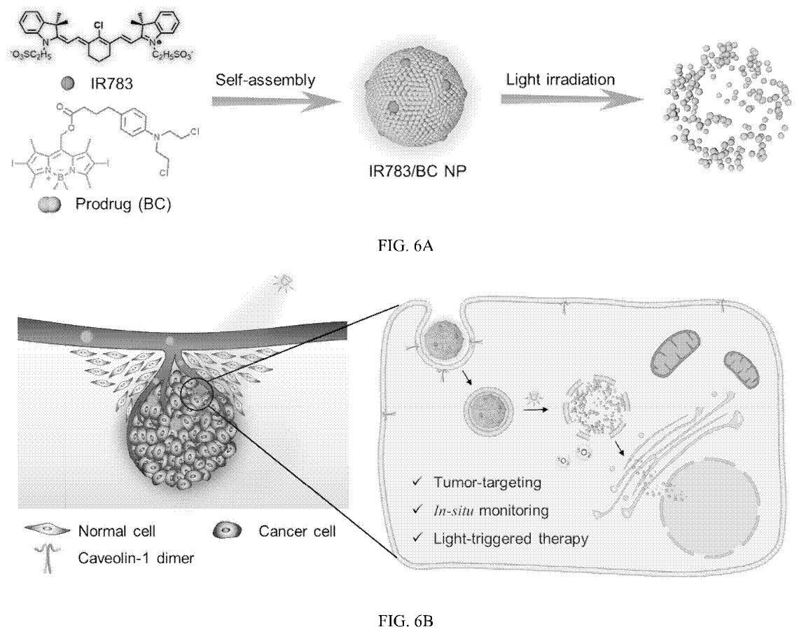

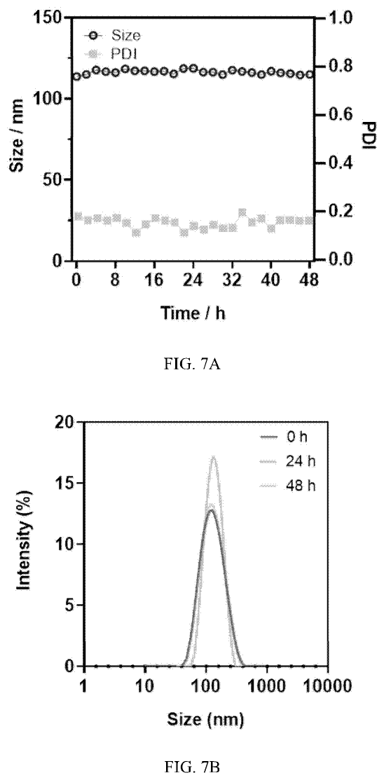

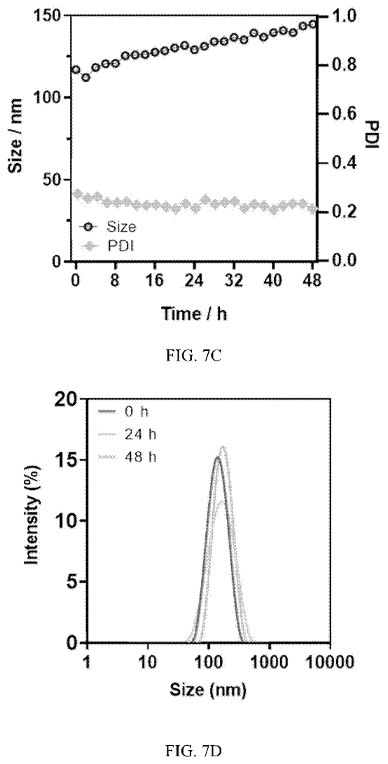

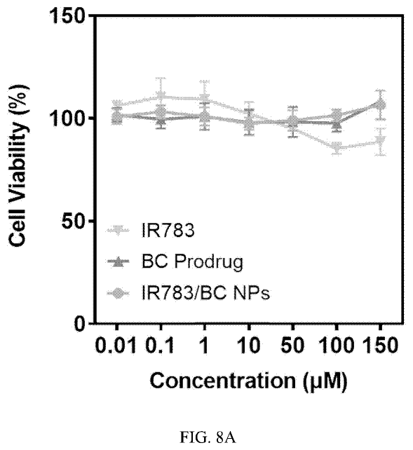

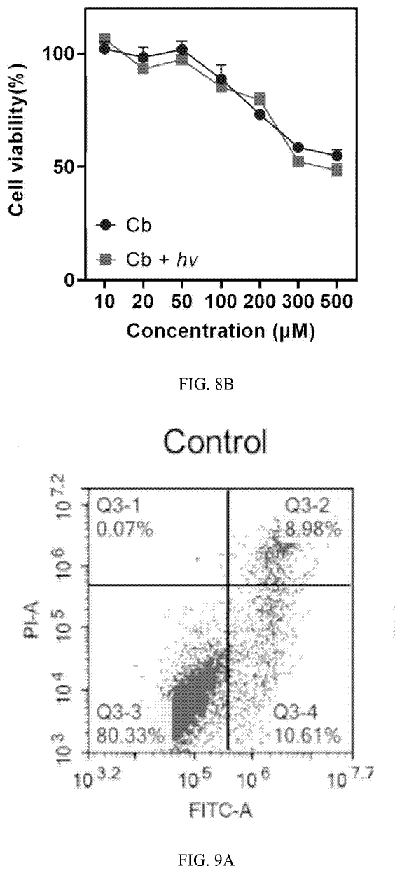

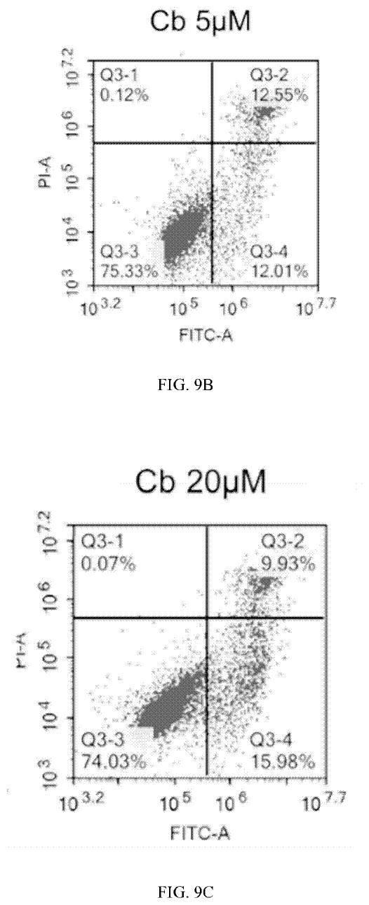

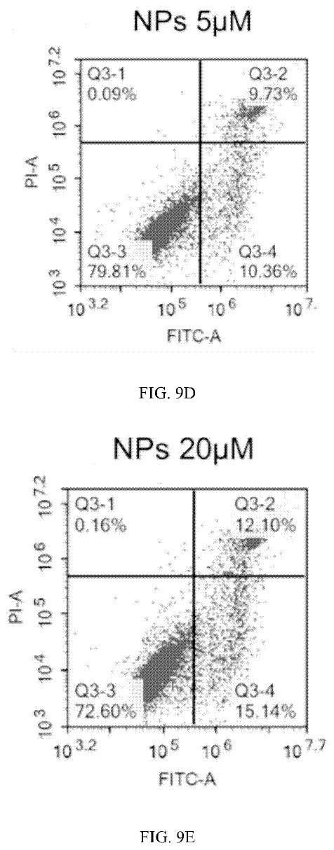

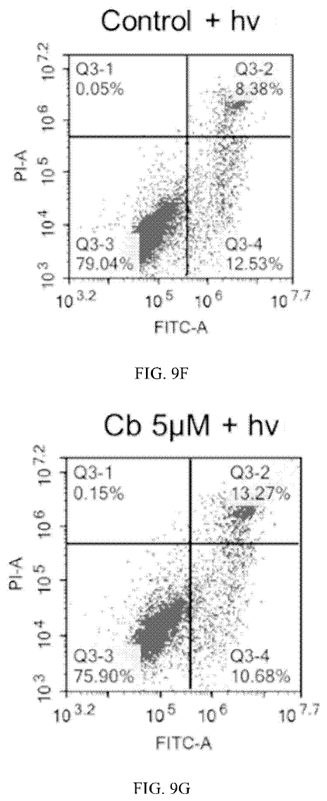

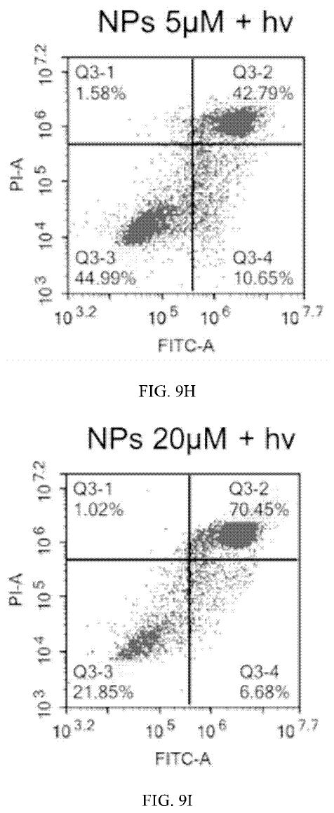

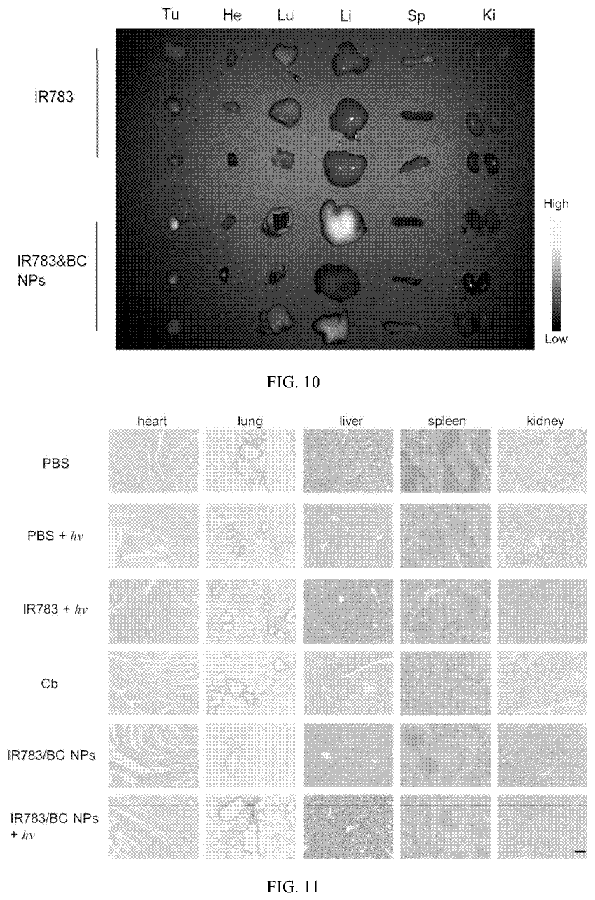

NPs after light irradiation (530 nm, 50 mW/cm²). (FIG. 2C) Size distribution of IR783/BC NPs under light irradiation for 0, 3 and 5 min. (FIG. 2D) High performance liquid chromatography (HPLC) analysis of IR783/BC NPs upon light irradiation. (FIG. 2E) Quantitative analysis of IR783/BC degradation and Cb release. (n=3) (FIG. 2F) UV-vis absorption of IR783 and fluorescence spectra of free IR783 (FIG. 2G) and IR783/BC NPs (FIG. 2H) upon 530 nm light irradiation. The photocleavage process of BC prodrug (FIG. 21) and ROS-response mechanism of IR783 (FIG. 2J). UV-vis absorption of ICG/BC NPs (FIG. 2K) and IR820/BC NPs (FIG. 2L) upon 530 nm light irradiation. Fluorescence spectra of ICG/BC NPs (FIG. 2M) upon 530 nm light irradiation. The excitation wavelength is 680 nm.

FIGS. 3A-3G. In vitro cellular uptake, cytotoxicity and ¹O₂ generation. (FIG. 3A) Confocal laser scanning microscopy (CLSM) images of free IR783 and IR783/BC NPs uptake by HCT116 cells after 6-h incubation. Scale bar: 20 μm. (FIG. 3B) In vitro cytotoxicity test of free BC prodrug and IR783/BC NPs with/without light by MTT assay. *p<0.05, ***p<0.005. (FIG. 3C) Calcein-AM/PI staining of HCT116 cells after treatment with free BC, free IR783, IR783/BC NPs with/without light. Scale bar: 100 μm. (FIG. 3D) Fluorescent change of IR783/BC NPs solution containing SOSG as ¹O₂ sensor. (FIG. 3E) The ¹O₂ production of IR783, free BC and IR783/BC NPs in the presence of light. (FIGS. 3F-3G) CLSM images of intracellular ¹O₂ in HCT116 cells incubated with IR783/BC NPs at different concentrations with/without light irradiation. DCFH-DA was used as the indicator. Scale bar: 10 μm.

FIGS. 4A-4E. In vivo biodistribution and in-situ monitoring upon light. (FIG. 4A) Schematic illustration of the treatment process in HCT116 tumor-bearing mice. (FIG. 4B) Representative IVIS fluorescent images of the mice post-injection of free IR783 and IR783/BC NPs within 24 h (n=3). The white cycle represented the tumor site. (FIG. 4C) Quantitative analysis of biodistribution determined by IVIS in major organs and tumor. Tu, He, Lu, Sp, Li and Ki represented tumor, heart, lung, spleen, liver and kidney. (FIG. 4D) In-situ fluorescence imaging of the mice 24 h post-injection of IR783/BC NPs with light irradiation at the tumor site for various durations (n=3). **p<0.01. (FIG. 4E) Quantitative analysis of fluorescence intensity in the tumor region exposed to various light irradiation durations.

FIGS. 5A-5H. Antitumor efficacy in HCT116 tumor model. (FIG. 5A) Schematic illustrating treatment schedule for inhibiting subcutaneous tumor growth. (FIG. 5B) Tumor volume change after treatment with PBS, IR783, Cb and IR783/BC NPs with/without light irradiation (n=4). **p<0.01. (FIGS. 5C-5D) Photos of mice with subcutaneous tumor, (FIG. 5E) tumor weight, (FIG. 5F) photos of tumors at Day 14 after different treatments as indicated. (FIG. 5G) Change of body weight of the mice during the treatments. (FIG. 5H) Representative H&E staining, TUNEL staining and immunobiological staining of CAV-1 protein of the tumor sections. Scale bar: 200 μm (H&E) 50 μm (TUNEL) and 50 μm (CAV-1), respectively.

FIGS. 6A-6B. Schematic illustration of photoresponsive IR783/BC NP and its therapeutic effect upon light irradiation. (FIG. 6A) Self-assembly of IR783/BC NP and its dissociation upon light irradiation. (FIG. 6B) The CAV-1-mediated cellular uptake of IR783/BC NP by a HCT116 cell and light-triggered in-situ monitorable cancer therapy.

FIGS. 7A-7D. Stability of IR783/BC NPs in (FIGS. 7A-7B) DMEM medium and (FIGS. 7C-7D) 10% fetal bovine serum contained DMEM medium in 37° C. for 48 h.

FIGS. 8A-8B. (FIG. 9A) Cell viability of HUVECs treated with IR783, BC and IR783/BC NPs in dark. (FIG. 9B) Cytotoxicity of chlorambucil towards HCT116 cells with/without light irradiation (530 nm, 50 mW/cm², 10 min).

FIGS. 9A-9I Apoptosis study of HCT116 cells treated with Cb and IR783/BC NPs with & without light irradiation (530 nm, 50 mW/cm^{2,} 10 min).

FIG. 10 Representative fluorescent images of the tumor and major organs 24 h after the injections of IR783 and IR783/BC NPs, respectively.

FIG. 11. H&E staining of major organs sections of the mice after different treatments. Scare bar: 200 μm.

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” is used, these compositions contain the stated amount of the ingredient with a variation (error range) of 0-10% around the value (X±10%). In other contexts the term “about” provides a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, the term “subject” refers to an animal, needing or desiring delivery of the benefits provided by a therapeutic compound. The animal may be for example, humans, pigs, horses, goats, cats, mice, rats, dogs, apes, fish, chimpanzees, orangutans, guinea pigs, hamsters, cows, sheep, birds, chickens, as well as any other vertebrate or invertebrate. These benefits can include, but are not limited to, the treatment of a health condition, disease or disorder; prevention of a health condition, disease or disorder; immune health; enhancement of the function of an organ, tissue, or system in the body. The preferred subject in the context of this invention is a human. The subject can be of any age or stage of development, including infant, toddler, adolescent, teenager, adult, or senior.

As used herein, the terms “therapeutically-effective amount,” “therapeutically-effective dose,” “effective amount,” and “effective dose” are used to refer to an amount or dose of a compound or composition that, when administered to a subject, is capable of treating or improving a condition, disease, or disorder in a subject or that is capable of providing enhancement in health or function to an organ, tissue, or body system. In other words, when administered to a subject, the amount is “therapeutically effective.” The actual amount will vary depending on a number of factors including, but not limited to, the particular condition, disease, or disorder being treated or improved; the severity of the condition; the particular organ, tissue, or body system of which enhancement in health or function is desired; the weight, height, age, and health of the patient; and the route of administration.

As used herein, the term “treatment” refers to eradicating, reducing, ameliorating, or reversing a sign or symptom of a health condition, disease or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

As used herein, “preventing” a health condition, disease, or disorder refers to avoiding, delaying, forestalling, or minimizing the onset of a particular sign or symptom of the condition, disease, or disorder. Prevention can, but is not required, to be absolute or complete; meaning, the sign or symptom may still develop at a later time. Prevention can include reducing the severity of the onset of such a condition, disease, or disorder, and/or inhibiting the progression of the condition, disease, or disorder to a more severe condition, disease, or disorder.

In some embodiments of the invention, the method comprises administration of multiple doses of the compounds of the subject invention. The method may comprise administration of 1, 2, 3, 4, 5, 6, 7, 8, 9, 10, 15, 20, 25, 30, 35, 40, or more therapeutically effective doses of a composition comprising the compounds of the subject invention as described herein. In some embodiments, doses are administered over the course of 1 day, 2 days, 3 days, 4 days, 5 days, 6 days, 7 days, 10 days, 14 days, 21 days, 30 days, or more than 30 days. Moreover, treatment of a subject with a therapeutically effective amount of the compounds of the invention can include a single treatment or can include a series of treatments. It will also be appreciated that the effective dosage of a compound used for treatment may increase or decrease over the course of a particular treatment. Changes in dosage may result and become apparent from the results of diagnostic assays or imaging techniques for detecting tumor sizes known in the art. In some embodiments of the invention, the method comprises administration of the compounds at several time per day, including but not limiting to 2 times per day, 3 times per day, and 4 times per day.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, a “pharmaceutical” refers to a compound manufactured for use as a medicinal and/or therapeutic drug.

As used herein, a “photoresponsive prodrug” is a compound that can be converted by light irradiation to a specific drug compound or a pharmaceutically acceptable salt of such a compound. Photoresponsive prodrugs are compounds that are covalently bonded to a photocleavable group.

As used herein, a “photoresponsive group” or “photocleavable group” is a chemical group that can be removed or separated with light irradiation via photocleavage reaction.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

Preparation of Prodrug NPs with a Dye and Compositions thereof

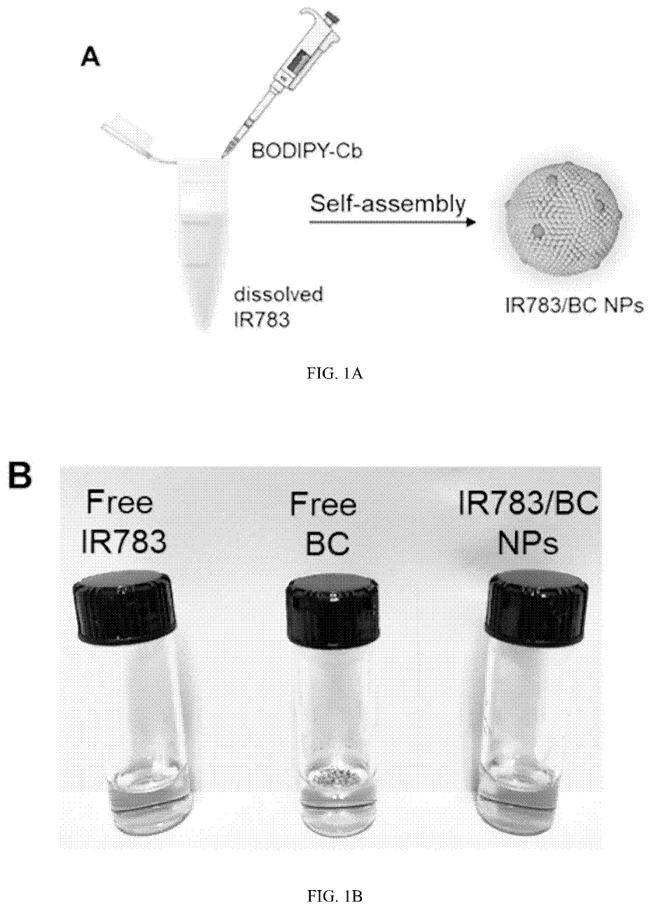

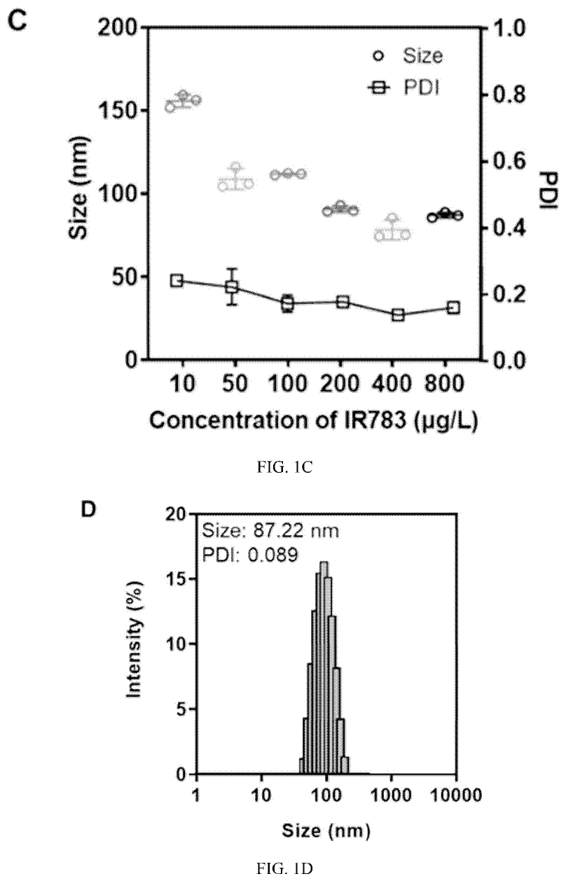

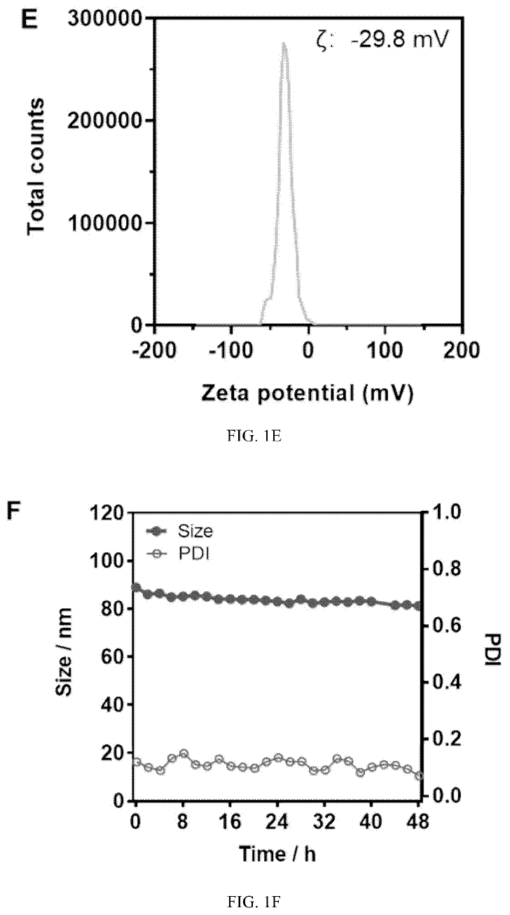

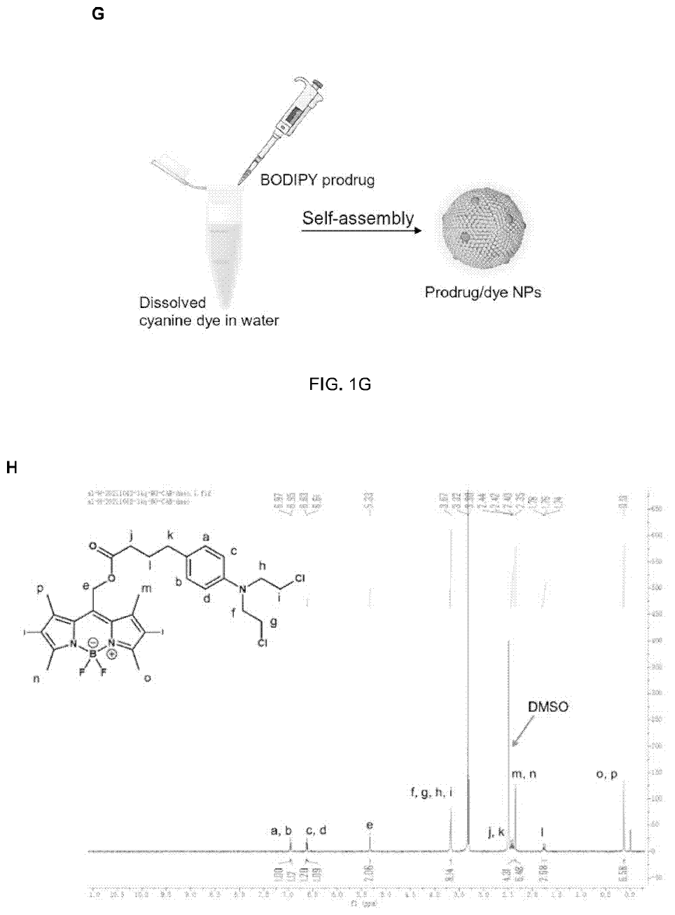

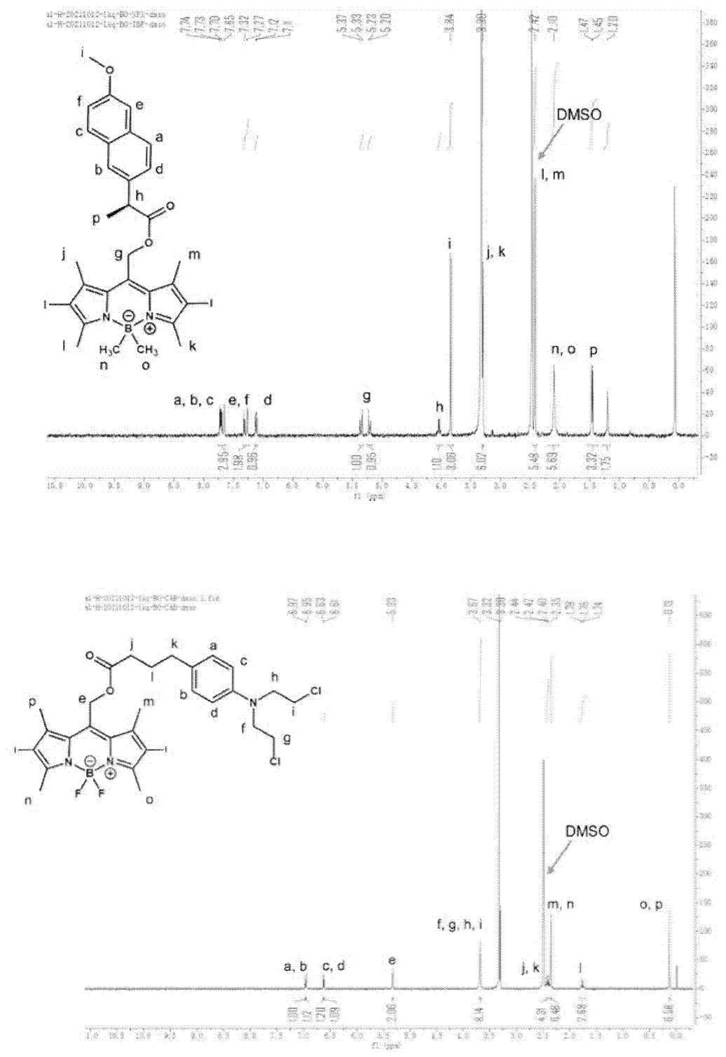

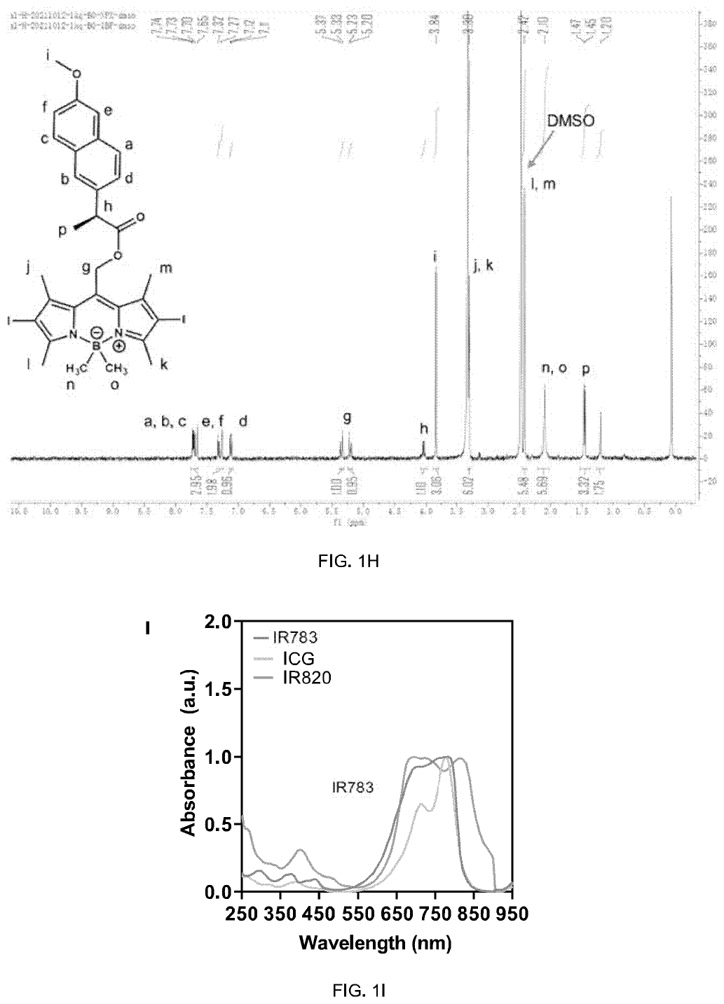

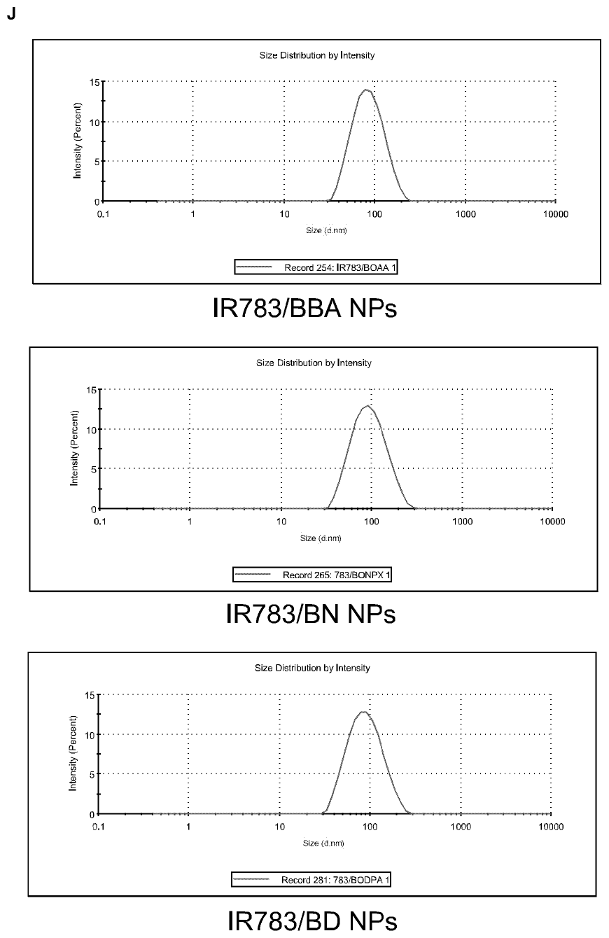

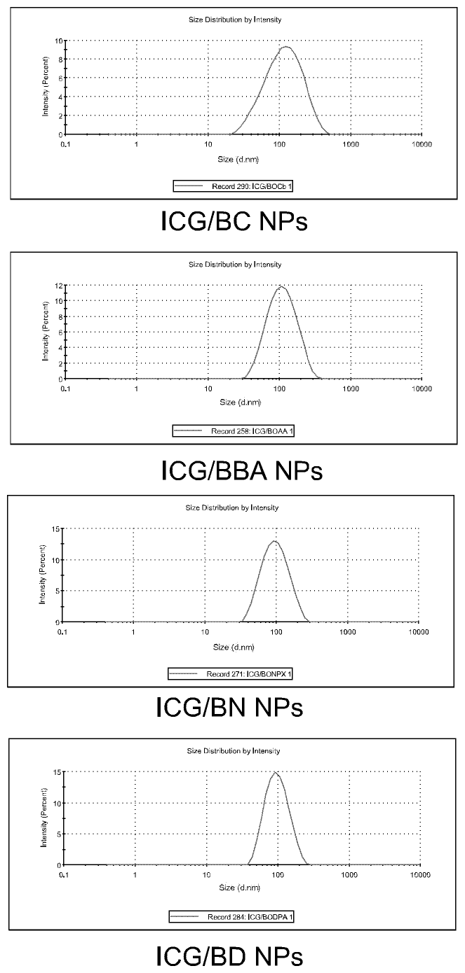

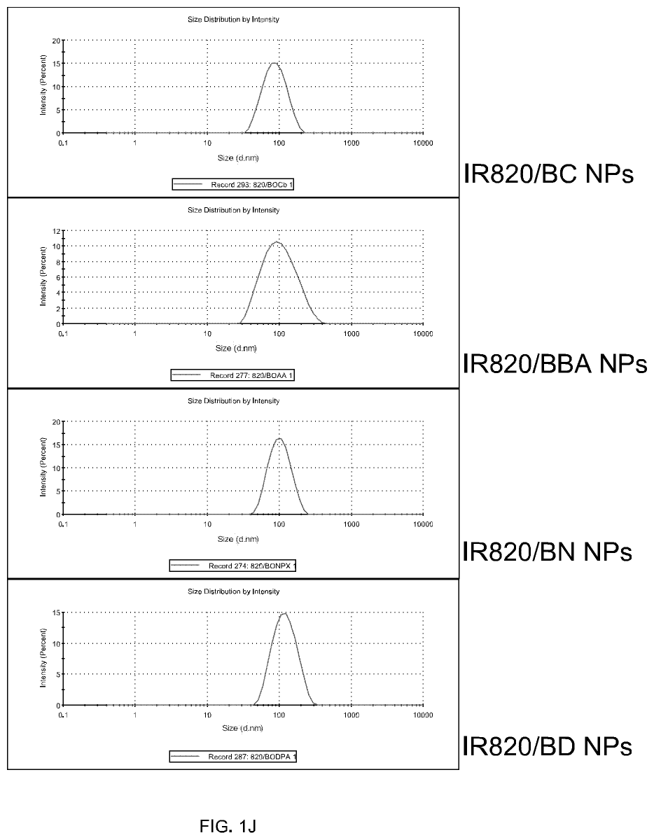

In certain embodiments, a photo-responsive nanoparticle can be synthesized using a therapeutic compound and a dye. In certain embodiments, the therapeutic compound can be boron-dipyrromethene-chlorambucil (BC) according to formula (Ia), boron-dipyrromethene-naproxen (BN) according to formula (Ib), boron-dipyrromethene-benzyloxycinnamic acid (BBA) according to formula (Ic), and/or boron-dipyrromethene-dopamine (BD) according to formula (Id) assembled with a dye. The NMR spectra of BC, BN, BBA and SD are shown in FIG. 1H. In certain embodiments, the dye can be a near-infrared dye, such as, for example, IR783 (according to formula (II)), IR820 (according to formula (III)), or Indocyanine green (ICG) (according to formula (IV)). IR783, IR820 and ICG are near-infrared cyanine dyes, which absorption spectra are shown in FIG. 11.

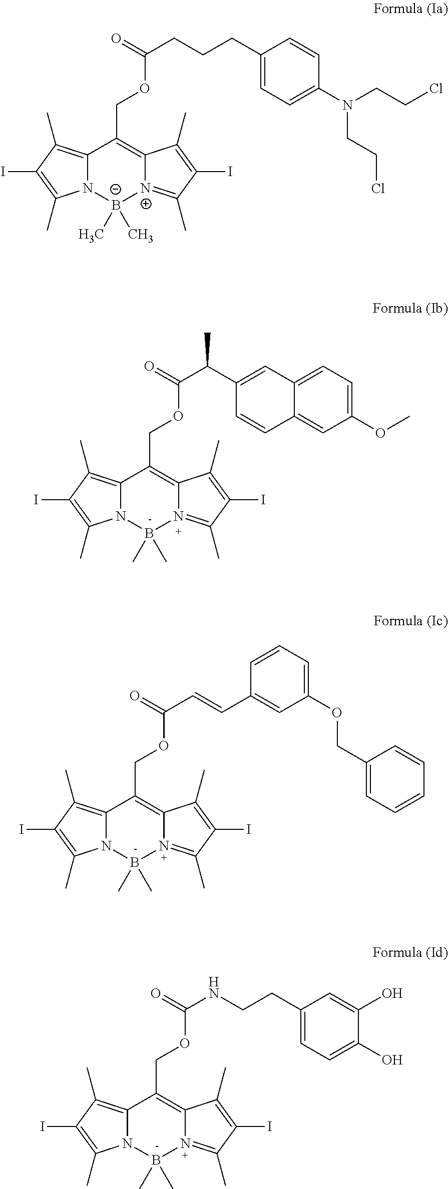 [SMILES: CC1=C(I)C(C)=[N+]2C1=C(COC(=O)CCCc1ccc(N(CCCl)CCCl)cc1)c1c(C)c(I)c(C)n1[B-]2(C)C ; COc1ccc2cc([C@H](C)C(=O)OCC3=C4C(C)=C(I)C(C)=[N+]4[B-](C)(C)n4c(C)c(I)c(C)c43)ccc2c1 ; CC1=C(I)C(C)=[N+]2C1=C(COC(=O)/C=C/c1cccc(OCc3ccccc3)c1)c1c(C)c(I)c(C)n1[B-]2(C)C ; CC1=C(I)C(C)=[N+]2C1=C(COC(=O)NCCc1ccc(O)c(O)c1)c1c(C)c(I)c(C)n1[B-]2(C)C]

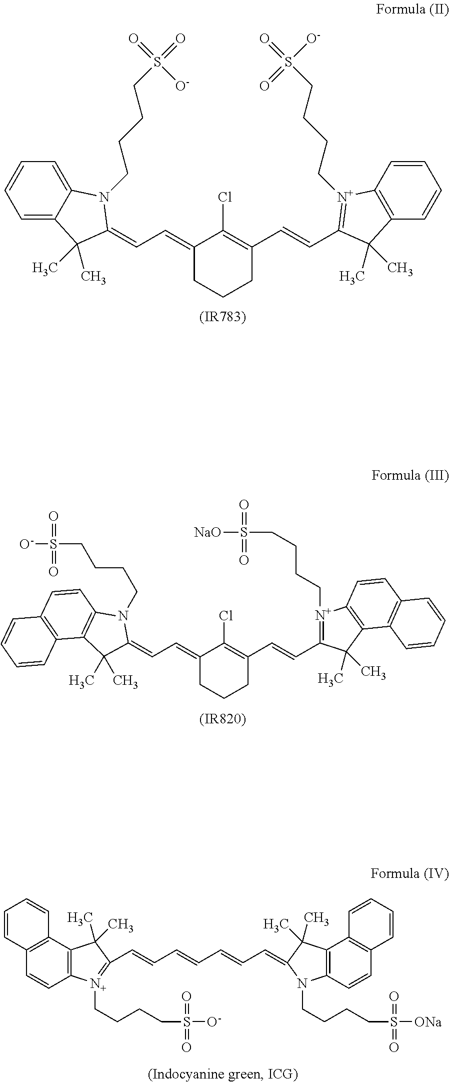

In certain embodiments, BC can be formed into a nanoparticle with a dye. In certain embodiments, BC can be dissolved in a solvent, such as, for example DMSO (20 μL, 10 mg/mL), DMG, or ethanol, and added to a solution containing IR783 (at a concentration of about 400 μg/mL), optionally under vigorous vortex. The solution can be centrifuged once to remove the precipitate, and the resulting precipitate can be re-suspended in sterile aqueous solutions, such as, for example, PBS, water, or cell culture media. The nanoparticle comprising suitable prodrug compounds (e.g., BC, BN, BBA and/or BD) and suitable dyes (e.g., IR783, IR820, and/or ICG) can be prepared with the same procedure as BC and IR783.

In preferred embodiments, the compositions and methods according to the subject invention utilize a prodrug, such as, for example, BC. The prodrug compounds (e.g., BC) may be in a purified form. Prodrug compounds may be added to compositions at concentrations of 0.01 to 99.99% by weight (wt %), preferably 50 to 99.99 wt %, and more preferably 90 to 99.99 wt %. In another embodiment, a purified prodrug compound may be in combination with an acceptable carrier, in that prodrug compound may be presented at concentrations of 0.001 to 50% (v/v), preferably, 0.01 to 50% (v/v).

In one embodiment, the subject compositions are formulated as an orally-consumable product, such as, for example a food item, capsule, pill, or drinkable liquid. An orally deliverable pharmaceutical is any physiologically active substance delivered via initial absorption in the gastrointestinal tract or into the mucus membranes of the mouth. The topic compositions can also be formulated as a solution that can be administered via, for example, injection, which includes intravenously, intraperitoneally, intramuscularly, intrathecally, or subcutaneously. In other embodiments, the subject compositions are formulated to be administered via the skin through a patch or directly onto the skin for local or systemic effects. The compositions can be administered sublingually, buccally, rectally, or vaginally. Furthermore, the compositions can be sprayed into the nose for absorption through the nasal membrane, nebulized, inhaled via the mouth or nose, or administered in the eye or ear.

Orally consumable products according to the invention are any preparations or compositions suitable for consumption, for nutrition, for oral hygiene, or for pleasure, and are products intended to be introduced into the human or animal oral cavity, to remain there for a certain period of time, and then either be swallowed (e.g., food ready for consumption or pills) or to be removed from the oral cavity again (e.g., chewing gums or products of oral hygiene or medical mouth washes). While an orally-deliverable pharmaceutical can be formulated into an orally consumable product, and an orally consumable product can comprise an orally deliverable pharmaceutical, the two terms are not meant to be used interchangeably herein.

Orally consumable products include all substances or products intended to be ingested by humans or animals in a processed, semi-processed, or unprocessed state. This also includes substances that are added to orally consumable products (particularly food and pharmaceutical products) during their production, treatment, or processing and intended to be introduced into the human or animal oral cavity.

Orally consumable products can also include substances intended to be swallowed by humans or animals and then digested in an unmodified, prepared, or processed state; the orally consumable products according to the invention therefore also include casings, coatings, or other encapsulations that are intended to be swallowed together with the product or for which swallowing is to be anticipated.

In one embodiment, the orally consumable product is a capsule, pill, syrup, emulsion, or liquid suspension containing a desired orally deliverable substance. In one embodiment, the orally consumable product can comprise an orally deliverable substance in powder form, which can be mixed with water or another liquid to produce a drinkable orally-consumable product.

In some embodiments, the orally-consumable product according to the invention can comprise one or more formulations intended for nutrition or pleasure. These particularly include baking products (e.g., bread, dry biscuits, cake, and other pastries), sweets (e.g., chocolates, chocolate bar products, other bar products, fruit gum, coated tablets, hard caramels, toffees and caramels, and chewing gum), alcoholic or non-alcoholic beverages (e.g., cocoa, coffee, green tea, black tea, black or green tea beverages enriched with extracts of green or black tea, Rooibos tea, other herbal teas, fruit-containing lemonades, isotonic beverages, soft drinks, nectars, fruit and vegetable juices, and fruit or vegetable juice preparations), instant beverages (e.g., instant cocoa beverages, instant tea beverages, and instant coffee beverages), meat products (e.g., ham, fresh or raw sausage preparations, and seasoned or marinated fresh meat or salted meat products), eggs or egg products (e.g., dried whole egg, egg white, and egg yolk), cereal products (e.g., breakfast cereals, muesli bars, and pre-cooked instant rice products), dairy products (e.g., whole fat or fat reduced or fat-free milk beverages, rice pudding, yoghurt, kefir, cream cheese, soft cheese, hard cheese, dried milk powder, whey, butter, buttermilk, and partly or wholly hydrolyzed products containing milk proteins), products from soy protein or other soy bean fractions (e.g., soy milk and products prepared thereof, beverages containing isolated or enzymatically treated soy protein, soy flour containing beverages, preparations containing soy lecithin, fermented products such as tofu or tempeh products prepared thereof and mixtures with fruit preparations and, optionally, flavoring substances), fruit preparations (e.g., jams, fruit ice cream, fruit sauces, and fruit fillings), vegetable preparations (e.g., ketchup, sauces, dried vegetables, deep-freeze vegetables, pre-cooked vegetables, and boiled vegetables), snack articles (e.g., baked or fried potato chips (crisps) or potato dough products and extrudates on the basis of maize or peanuts), products on the basis of fat and oil or emulsions thereof (e.g., mayonnaise, remoulade, and dressings), other ready-made meals and soups (e.g., dry soups, instant soups, and pre-cooked soups), seasonings (e.g., sprinkle-on seasonings), sweetener compositions (e.g., tablets, sachets, and other preparations for sweetening or whitening beverages or other food). The present compositions may also serve as semi-finished products for the production of other compositions intended for nutrition or pleasure.

The subject composition can further comprise one or more pharmaceutically acceptable carriers, and/or excipients, and can be formulated into preparations, for example, solid, semi-solid, liquid, or gaseous forms, such as tablets, capsules, powders, granules, ointments, solutions, suppositories, injections, inhalants, and aerosols.

The term “pharmaceutically acceptable” as used herein means compatible with the other ingredients of a pharmaceutical composition and not deleterious to the recipient thereof.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance or with the composition, carrier or excipient use in the subject compositions may be contemplated.

In one embodiment, the compositions of the subject invention can be made into aerosol formulations SO that, for example, it can be nebulized or inhaled. Suitable pharmaceutical formulations for administration in the form of aerosols or sprays are, for example, powders, particles, solutions, suspensions or emulsions. Formulations for oral or nasal aerosol or inhalation administration may also be formulated with carriers, including, for example, saline, polyethylene glycol or glycols, DPPC, methylcellulose, or in mixture with powdered dispersing agents or fluorocarbons. Aerosol formulations can be placed into pressurized propellants, such as dichlorodifluoromethane, propane, nitrogen, fluorocarbons, and/or other solubilizing or dispersing agents known in the art. Illustratively, delivery may be by use of a single-use delivery device, a mist nebulizer, a breath-activated powder inhaler, an aerosol metered-dose inhaler (MDI), or any other of the numerous nebulizer delivery devices available in the art. Additionally, mist tents or direct administration through endotracheal tubes may also be used.

In one embodiment, the compositions of the subject invention can be formulated for administration via injection, for example, as a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono-or diglycerides, and fatty acids, including oleic acid. One illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

In one embodiment, the compositions of the subject invention can be formulated for administration via topical application onto the skin, for example, as topical compositions, which include rinse, spray, or drop, lotion, gel, ointment, cream, foam, powder, solid, sponge, tape, vapor, paste, tincture, or using a transdermal patch. Suitable formulations of topical applications can comprise in addition to any of the pharmaceutically active carriers, for example, emollients such as carnauba wax, cetyl alcohol, cetyl ester wax, emulsifying wax, hydrous lanolin, lanolin, lanolin alcohols, microcrystalline wax, paraffin, petrolatum, polyethylene glycol, stearic acid, stearyl alcohol, white beeswax, or yellow beeswax. Additionally, the compositions may contain humectants such as glycerin, propylene glycol, polyethylene glycol, sorbitol solution, and 1,2,6 hexanetriol or permeation enhancers such as ethanol, isopropyl alcohol, or oleic acid.

### Methods of Using Compounds of the Subject Invention

In certain embodiments, a prodrug and dye nanoparticle, such as, for example, IR783/BC or derivatives thereof can be administered to a subject. Any means of administration that can permit a nanoparticle to contact cells, including, for example, orally, intravenously, intraperitoneally, intramuscularly, intrathecally, or subcutaneously are envisioned in the subject methods.

In certain embodiments, after administration, a prodrug and dye nanoparticle, such as, for example, IR783/BC NPs can release, the therapeutic compound, such as, for example, chlorambucil upon light irradiation of the subject and/or the nanoparticle. In certain embodiments, the wavelength of the light can be about 100 nm to about 1000 nm, about 200 nm to about 700 nm, about 350 nm to about 550 nm, or, preferably, about 530 nm. In certain embodiments, the subject and/or prodrug can be irradiated for about 1 s to about 24 hours, about 10 s to about 12 hours, about 15 s to about 1 hour, about 30 s to about 30 min, about 45 s to about 10 min, about 1 min to about 5 min, or about 5 min. In certain embodiments, an entire subject can be irradiated. Alternatively, a specific portion of a subject can be irradiated, such as, for example, leg, arm, wrist, chest, abdomen, neck, or calf. In certain embodiments, the subject can be irradiated before, during, or after administration of the compounds or compositions of the subject invention. In certain embodiments, the irradiation of the nanoparticle and/or subject can occur at least 1 s, 2 s, 5 s, 10 s, 15 s, 30 s, 45 s, 1 min, 2 min, 5 min, 10 min, 15 min, 30 min, 45 min, 1 hour, 2 hour, 5 hours, or 10 hours after administration of the compounds or compositions of the subject invention. In certain embodiments, the subject and/or nanoparticle can be irradiated after the nanoparticle is at a specific location, such as, for example, tumor, organ, tissue, or blood-brain barrier. In certain embodiments, the irradiance can be about 1 mW/cm² to about 1000 mW/cm², 5 mW/cm² to about 500 mW/cm², 10 mW/cm² to about 250 mW/cm², 15 mW/cm² to about 100 mW/cm², 25 mW/cm² to about 75 mW/cm², or about 50 mW/cm². The light can be delivered by any natural or artificial source capable of providing light at the wavelength and irradiance for the described amount of time, including, for example, incandescent, halogen, fluorescent, or light emitting diode (LED). In certain embodiments, under 530 nm light irradiation with relative low irradiance (50 mW/cm², 5 min), IR783/BC NPs can be completely decomposed due to the cleavage of BC prodrug, accompanied by the release of chlorambucil (FIG. 6A). In certain embodiments, the nanoparticle can emit fluorescence upon excitation that can be utilized as the indicator of the location of the nanoparticle.

In preferred embodiments, the subject compound can be irradiated to remove the photocleavable group and release the therapeutic agent. The removal rate of the photocleavable group can depend on the wavelength and intensity of the incident radiation, as well as the physical and chemical properties of the photocleavable group itself. In certain embodiments, five minutes or less of light irradiation can remove almost 100% of the photocleavable groups.

In certain embodiments, the subject nanoparticles can have a prodrug loading capacity of up to about 80%, about 85%, about 90%, about 95%, about 98%, or, preferably, about 99%. In certain embodiments, the dye, such as, for example, IR783 co-assembled in the photoresponsive prodrug-based nanoparticles can provide versatile functions by serving as: 1) stabilizer that can be degraded after light irradiation; 2) targeting moiety with sulfate groups that enhance the caveolin-1 (CAV-1) mediated transcytosis in tumor; 3) fluorescent imaging agent for in-situ monitoring of nanoparticle disassembly (FIG. 6B) [29-31]. Thus, IR783/BC NPs can achieve tumor-targeting, fluorescent imaging, and light-triggered drug release in cancer treatment. In certain embodiments, administration of the nanoparticles of the subject invention can eliminate tumors in subjects upon light irradiation.

In certain embodiments, light irradiation, such as, for example, 530 nm green-light irradiation, the nanoparticle, including, for example, IR783/BC NPs dissociated, releasing anti-cancer agent chlorambucil and generating ¹O₂, exhibiting its light-triggered anti-tumor effect. In certain embodiments, singlet oxygen can enhance the anti-tumor effect via the photodynamic therapy and/por cause the degradation of the dye to promote the disassembly of nanoparticles. In certain embodiments, the near-infrared (NIR) fluorescence emission of IR783 in the nanoparticles can display an “ON-to-OFF” pattern while applying light irradiation, which means that the IR783 fluorescence gradually decreases with light irradiation. Such phenomenon can enable in-situ fluorescence imaging of light-triggered dissociation of nanoparticles. In certain embodiments, the sulfate groups of IR783 can enable the enhanced tumor accumulation of IR783/BC NPs by CAV-1-mediated transcytosis, which is supported by references [27, 28] and verified by the cellular uptake study of IR783/BC NPs.

In certain embodiments, IR783 can present versatile functions by serving as stabilizer, tumor-targeting moiety and imaging agent in the subject compositions and methods. IR783 exhibits such functions because of its integration in the photoresponsive system, for example, the photoresponsive prodrug-based nanoparticles.

### Materials And Methods

### Materials and Instruments

Indocyanine IR783 dye was purchased from Tokyo Chemical Industry (TCI) Co., Ltd (Tokyo, Japan). IR820 dye was purchased from Sigma-Aldrich (St. Louis, MO, USA). ICG dye was purchased from Sigma-Aldrich (St. Louis, MO, USA). Chlorambucil (Cb) was purchased from J&K Scientific (Beijing, China). Naproxen was purchased from BLDpharm (Shanghai, China). Benzyloxycinnamic acid was purchase form BLDpharm (Shanghai, China). Dopamine was purchased from BLDpharm (Shanghai, China). Dimethyl sulfoxide (DMSO) was purchased from DUKSAN Pure Chemicals (Ansan, South Korea). Fetal bovine serum (FBS), RMPI 1640, MTT, penicillin-streptomycin were purchased from Gibco (Thermo Fisher Scientific, Waltham, MA, USA). Phosphate buffer saline (PBS) was purchased from Sigma-Aldrich (St. Louis, MO, USA). All chemicals were used directly without further purification. Deionized water was used to prepare all aqueous solutions. The UV-vis absorption spectra and cell viability were measured by a multi-mode microplate reader (SpectraMax® M4, Molecular Devices LLC, San Jose, CA, USA) using quartz cuvettes with 1-cm path length (Starna Scientific Ltd., UK). Size and zeta potential of nanoparticles (DLS) were measured by Nano ZS90 (Malven Instrument, Southborough, MA, USA). TEM images were obtained by CM100 Transmission Electron Microscope (Philips, Nederland). The product after photolysis was quantitatively determined by 1260 Infinity II HPLC (Agilent Technologies, Santa Clara, CA, USA). The excitation light source for photolysis and cell study was green-light LED (530 nm) (Mightex, Pleasanton, CA, USA). The excitation light source for in vivo study was green-light laser (530 nm) (Laserwave Co. Ltd., Beijing, China). The light irradiance was determined by PM100 USB power and energy meter (THORLABS Inc., Newton, NJ, USA) with S142C integrating sphere photodiode power sensor (Si 350-1100 nm, THORLABS). The NMR spectra were measured by Bruker AV300 NMR spectrometer (400 MHZ) (Billerica, MA, USA).

### Screening of Different Concentrations of IR783 for Co-Assembly

BODIPY-Cb (20 μL, 10 mg/mL, DMSO) was added dropwise (20 μL per 10 sec) to a group of 300 μL aqueous solution containing gradient concentrations of IR783 (10, 50, 100, 200, 400, and 800 μg/mL) under vigorous vortex, respectively. Then the particle size and PDI of each group were measured by DLS.

Preparation of IR783 BC NPs and other Prodrug Dye NPs

BODIPY-Cb was dissolved in DMSO (20 μL, 10 mg/mL), and added dropwise to a 300 μL aqueous solution containing IR783 (400 μg/mL) under vigorous vortex. The solution was centrifuged once (2000×g, 10 min, 4° C.) to remove the precipitate. Then the supernatant was centrifuged twice (30,000×g, 30 min, 4° C.), and the resulting precipitate was re-suspended in 300 μL of sterile PBS. Other prodrug/dye NPs were prepared following the same procedure as above. The contents of IR783 and BC in the resulted nanoparticle solution were determined by HPLC analysis. Their loading capacity and encapsulation efficiency were calculated as follow:

$$Loading capacity ( \% ) = \frac{weight of loaded IR 783 or BC}{weight of loaded nanoparticles} \times 100 \% \\ Encapsulation efficiency ( \% ) = \frac{weight of loaded IR 783 or BC}{weight of fed IR 783 or BC} \times 100 \%$$

### Stability of IR783 BC NPs

IR783/BC NPs (50 μM) were dissolved in water, PBS, RPMI 1640 cell culture medium, or FBS-contained medium at 37° C. in plastic cuvette shielded from light separately. The particle size and PDI were measured every 2 h by DLS within a total period of 48 h.

### Photocleavage of Prodrug in IR783 BC NPs

500 μL of the nanoparticle solution (50 μM) was irradiated by the LED light (530 nm, 50 mW/cm²) for different time periods (1, 2, 3, 5, 7, and 10 min). The irradiance of the LED irradiation was measured with a power and energy meter. After the irradiation, the size, morphology, and content of the solutions were analyzed by DLS, TEM and HPLC, respectively.

### Spectrum Study of IR783 Under Light Irradiation

The degradation of IR783 was demonstrated by spectroscopy. The IR783/BC NP solution was irradiated for different time periods (530 nm, 50 mW/cm², 0-60 s). At different time points, the absorbance and fluorescence were measured by spectrometers without any dilution. In the case of IR820/BC NP and ICG/BC NP, irradiation was performed at 530 nm for different time periods between 0 to 30 s.

### Cell Culture

HCT116 cells were cultured in RPMI-1640 (Gibco) supplemented with 10% FBS (Gibco) and 100 units/mL antibiotics (Penicillin-Streptomycin, Gibco) at 37° C. in a 5% CO₂ humidified atmosphere. HUVECs cells were cultured in DMEM (Gibco) supplemented with 10% FBS (Gibco) and 100 units/mL antibiotics (Penicillin-Streptomycin, Gibco) at 37° C. in a 5% CO₂ humidified atmosphere.

### Singlet Oxygen Detection

Singlet oxygen generation was measured both in solutions and cells using SOSG and DCFH-DA as indicator, respectively. Briefly, free IR783, free BC, IR783/BC NPs (at the equivalent concentration of BC at 1 μM) was mixed with SOSG probe (5 μM) in PBS. The solutions were exposed to light irradiation and the fluorescence intensity was detected at predetermined time intervals (Ex. 488 nm). Besides, the intracellular ¹O₂ generation was detected by CLSM. HCT116 cells were seeded in confocal dishes (Corning, 200350, Cell Culture-Treated, Corning, NY) at a density of 10000 cells/well and treated with different formulations for 6 h. Subsequently, the cells were washed with PBS for 3 times and incubated with DCFH-DA (10 μM) for 30 min. The cells were irradiated by LED (530 nm, 50 mW/cm², 5 min) for the light irradiation groups. The intracellular fluorescence was observed by CLSM imaging to evaluate the ¹O₂ generation.

### Cellular Uptake Study

Cellular uptake of free IR783 in solutions or IR783/BC NPs was investigated by using CLSM imaging. In brief, HCT116 cells were seeded in confocal dishes at a density of 10000 cells/well and treated with different formulations for 6 h in dark. The cells were washed by PBS for 3 times and stained with Hoechst 33342. Then the cells were directly observed by CLSM imaging.

### Cytotoxicty Study

The cell viability was evaluated by MTT assay. HCT116 cells were incubated in 96-well microliter plates for 24 h. Then, gradient concentrations of BC prodrug or IR783/BC NPs in RPMI 1640 culture medium were added to the cells. After 6 h incubation, the residual BC or IR783/BC NPs were removed by fresh RPMI 1640 culture medium (100 μL). Then, the cells were irradiated by 530 nm LED light (50 mW/cm², 5 min) or incubated at room temperature without light. After another incubation for 24 h, MTT solution (10 μL, 10 mg/mL) was added to the cells to cultivate for 3 h. Next, the cell culture medium was discarded, and DMSO (100 μL) was then added. The absorbance was measured by a multi-mode microplate reader at 490 nm, 570 nm and 630 nm.

### Live Dead Staining and Apoptosis Detection

For live/dead staining, HCT116 cells were seeded in confocal dishes at a density of 10000 cells/well and treated with PBS, free IR783, free BC and IR783/BC NPs with an equivalent concentration of BC at 50 μM. For the irradiation groups, cells were washed by PBS after 6 h and irradiated by 530 nm LED for 5 min. Calcein-AM and PI were then added into the medium and the cells were observed by CLSM without any treatment.

For apoptosis study, HCT116 cells were seeded in 6-well plates at a density of 50000 cells/well and treated with free Cb or IR783/BC NPs at the equivalent concentration of Cb at 5 μM and 20 μM for 6 h. Then the cells were washed by PBS. The cells in the irradiation group were irradiated by light for 5 min. After 24-h incubation, cells were washed by PBS, collected by trypsin digestion, and stained with Annexin-V/FITC apoptosis kit. ACEA NovoCyte Quanteon flow cytometer (ACEA Biosciences, San Diego, CA, USA) was used for cell study.

### Animal Study

BALB/c nude mice were purchased from SLAC Experiment Animal Co., Ltd. (Shanghai, China) and kept in the Laboratory Animal Center of Fudan University (Shanghai, China). For the subcutaneous tumor model, male BALB/c nude mice were injected with 2×10⁷ HCT116 cells subcutaneously. The mice were then further kept in SPF condition for 5-7 days until tumors were observed.

### In Vivo Biodistribution

The biodistribution of IR783/BC NPs in the HCT116 tumor model was visualized by an in vivo fluorescence imaging system (Cailper PerkinElemer, Waltham, MA, United States). IR783, as a NIR fluorescent dye, was directly detected for living imaging. Mice were treated with free IR783 and IR783/BC NPs via intravenous injection with a dose of IR783 at 100 μg/kg. Fluorescence imaging was performed at 1, 6, 16, 20, and 24 h post injection. At 24 h, mice were euthanized and the tumors and major organs (heart, liver, spleen, lung, kidney) were excised for ex vivo imaging.

### In-Situ Monitoring of Light-Triggered Therapy

HCT116 tumor-bearing mice were intravenously injected with IR783/BC NPs with a dose of IR783 at 100 μg/kg. The mice were further kept in dark for 24 h, and light irradiation (530 nm, 100 mW/cm²) was applied on the tumor sites. Fluorescence imaging was performed before the irradiation and at 30, 60, 120, and 300 s post irradiation. The images and fluorescent intensity were recorded for analysis.

### In Vivo Antitumor Efficacy

The antitumor efficacy of IR783/BC NPs in the presence or absence of light was investigated in HCT116 tumor mouse model. The mice were randomly divided into six groups when the tumor size reached about 200 mm³. For different groups, mice were treated with different formulations via i.v. injection at Day 0, 4, 8, and 12: (1) PBS; (2) PBS+hv; (3) IR783+hv; (4) Cb; (5) IR783/BC NPs; (6) IR783/BC NPs+hv. For the irradiation group, light irradiations (100 mW/cm², 10 min) were performed 24 h post injection. The dose for each treatment was set as 5 mg/kg. Tumor sizes and body weights were measured in a 2-day interval, and the tumor volume was calculated as V=½×width²×length. At the end of the experiment (Day 14), all mice were euthanized, and the tumors and major organs were collected and sliced for H&E staining and immunohistochemical analysis.

### Statistical Analysis

All experiments were conducted three times or more independently (n≥3). Data were presented as the mean±standard deviation (SD). The one-way ANOVA-LSD and Independent-Samples t-test were adopted to determine the statistical significance of differences by Graphpad Prism 8.0 software (San Diego, CA).

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1

### Preparation and Characterization of IR783/BC NPS

The synthesis of photoresponsive BC prodrug followed the published method [32]. IR783 was reported to serve as stabilizer when forming nanoassemblies with hydrophobic drugs [27, 33]. As illustrated in FIG. 1A, the BC prodrug could co-assemble with IR783 when injecting the BC stock solution dropwise in the aqueous solution of IR783 by the flash nanoprecipitation method. The excessive IR783 in the solution was removed by centrifugation, and the IR783/BC nanoparticle was finally obtained as red-purple dispersion after resuspension in phosphate buffer saline (PBS) (FIG. 1B). We firstly optimized the feeding ratio of IR783 to BC prodrug by adjusting the concentrations of IR783 solution. When the concentration of IR783 solution was 400 μg/mL, the obtained nanoparticles showed both small diameter and low polydispersity index (PDI), indicating an optimized mass ratio (FIG. 1C). Dynamic light scattering (DLS) detected IR783/BC NPs as nanoassemblies with a hydrodynamic diameter of 87.22 nm and a PDI at 0.089 (FIG. 1D). The IR783/BC NPs was negatively charged at −29.8 mV, attributed to the sulfate groups of IR783 with negative charge (FIG. 1E). Moreover, we quantified the content of IR783 and BC prodrug in the nanoparticles by high-performance liquid chromatography (HPLC) and calculated the loading capacity and encapsulation efficiency of the two components. As shown in Table 1, we found that both the loading capacity and encapsulation efficiency of BC prodrug were remarkably high, reaching 98.85% and 84.37%, respectively, which demonstrated that the BC prodrug can efficiently assembled into nanoparticles in the present of IR783.

Subsequently, we investigated the stability of IR783/BC NPs in PBS, DMEM medium and FBS solution separately at 37° C. for two days in the dark environment. Under these conditions, IR783/BC NPs maintained their diameters and PDI, indicating the good stability of the prodrug nanoparticles (FIG. 1F and FIGS. 7A-7D).

TABLE 1
Loading capacity and encapsulation
efficiency of IR783 and BC in NPs.

| | Feeding | Weight after | Loading | Encapsulation |
|---|---|---|---|---|
| | weight (μg) | purification (μg) | Capacity | Efficiency |
| IR783 | 300 | 0.61 | 1.15% | 0.02% |
| BODIPY-Cb | 200 | 168.74 | 98.85% | 84.37% |

Further to IR783/BC NPs, Dynamic light scattering (DLS) detected the size distribution (FIG. 1J) and the PDI for other prodrug/dye NPs, as summarize below in Table 2.

TABLE 2
Size and PDI of prodrug/dye NPs.

| Dye | Prodrug | Size (nm) | PDI |
|---|---|---|---|
| IR783 | BC | 87.22 ± 2.05 | 0.089 ± 0.020 |
| | BBA | 73.45 ± 4.13 | 0.138 ± 0.023 |
| | BN | 80.31 ± 2.60 | 0.183 ± 0.021 |
| | BD | 79.59 ± 0.86 | 0.164 ± 0.025 |
| ICG | BC | 97.37 ± 0.68 | 0.248 ± 0.037 |
| | BBA | 84.58 ± 2.38 | 0.154 ± 0.021 |
| | BN | 76.50 ± 1.07 | 0.262 ± 0.002 |
| | BD | 87.62 ± 1.94 | 0.173 ± 0.034 |
| IR820 | BC | 77.12 ± 1.91 | 0.123 ± 0.034 |
| | BBA | 85.73 ± 0.66 | 0.230 ± 0.020 |
| | BN | 92.21 ± 1.27 | 0.144 ± 0.023 |
| | BD | 102.53 ± 1.17 | 0.208 ± 0.014 |

### Example 2

### Light-Triggered Nanoparticle Dissociation and Drug Release

For practical applications in drug delivery, the photoresponsive nanoparticles not only need to remain intact in the formulated solution and blood circulation, but also can intelligently respond to light for controlled drug release [34]. Therefore, the photoresponsive property of IR783/BC NPs was investigated by monitoring the size, morphology, and composition changes via DLS, TEM and HPLC testing, respectively. It was observed that the spherical IR783/BC NPs disassociated and formed both large aggregations and small fragments after light irradiation (FIGS. 2A and 2B). The hydrodynamic diameter of IR783/BC NPs remarkably increased after 530 nm light irradiation at 50 mW/cm², indicating the disassembly of the nanoparticles and the formation of large aggregates as well (FIG. 2C). Moreover, the release process of anticancer drug chlorambucil from IR783/BC NPs upon light irradiation (530 nm, 50 mW/cm²) was investigated by HPLC (FIGS. 2D and 2E). We noticed that the elution peak at 28.9 min corresponding to the BC prodrug decreased over time. A new broad peak at around 12.0 min corresponding to free chlorambucil gradually increased, indicating the photocleavage of BC prodrug and the release of free drug. Within 10-min irradiation, nearly 100% of BC prodrug was degraded and free chlorambucil was released. These results demonstrated that IR783/BC NPs exhibited both good stability in dark and excellent photocleavable ability upon light irradiation, confirming their potential as an ideal candidate for photoresponsive drug delivery.

The photoresponsive property of IR783/BC NPs was further investigated by UV-Vis-NIR spectrometry. As shown in FIG. 2F, the IR783/BC NPs without light irradiation displayed intense absorption peaks at around 530 nm and 780 nm, which are corresponding to the absorption of BC prodrug and IR783, respectively [32, 35]. Upon light irradiation using a 530 nm LED lamp (50 mW/cm²), the absorption peak at 780 nm disappeared within 20 s, indicating the degradation of IR783 in the nanoparticles upon the light irradiation. HPLC result also showed that IR783 in the nanoparticles degraded completely after 1-min light irradiation (FIG. 2E). It should be noted that the peak at 530 nm, corresponding to BC, did not change obviously, which indicated that the light-harvesting capability of BODIPY group of the prodrug was maintained after the light-triggered cleavage. Additionally, IR783/BC NPs showed broad NIR fluorescent emission in the region of 780-950 nm that belongs to the emission profile of IR783 dye [36]. The NIR emission displayed an “ON-to-OFF” pattern while applying 530 nm light irradiation on IR783/BC NPs, which also indicated the degradation of IR783 (FIG. 2H). Notably, we did not see similar phenomena while free IR783 was irradiated (FIG. 2G). Thus, the photoactivation of BC that generates reactive oxygen species should play a vital role in the light-triggered fluorescence deactivation. As the above results indicated, the photo-response mechanisms of BC prodrug and IR783 were shown in FIGS. 21-2J. The “ON-to-OFF” fluorescence switching pattern enabled in-situ monitoring of the photoresponsive process of IR783/BC NPs, including accumulation in tumors and light-triggered dissociation of nanoparticles.

Similarly, FIGS. 2K-2M showed that ICG and IR820 are decomposed upon light irradiation.

### Example 3

### Cellular Uptake, Cytotoxicity and Apoptosis Study

The intracellular uptake of IR783/BC NPs in human colon tumor HCT116 cells were evaluated by confocal laser scanning microscope (CLSM). This cancer cell line was reported to display a high expression of caveolae, which can facilitate the internalization of IR783/BC NPs [33, 37, 38]. As shown in FIG. 3A, after 6 h of incubation, the nanoparticles exhibited remarkably stronger intracellular red fluorescence which represented IR783. Based on previous literatures, the nanoparticles coated by sulfated indocyanines elicited caveolae-targeting effects, which was

confirmed by both immunohistochemistry and CAV-1 knockout assay [27]. In vitro cytotoxicity studies including the evaluation of biocompatibility and anti-proliferation efficiency of our system were investigated. Regarding the increasing concerns about utilizing the synthesized agents in biological systems, the cytotoxicity of IR783, BC prodrug and IR783/BC NPs in dark was evaluated for the biocompatibility evaluation. Negligible cytotoxicity was observed when the human umbilical vein endothelial cells (HUVECs) were incubated with IR783, BC or IR783/BC NPs separately in dark for 24 h with a high concentration up to 100 μM, indicating the good biocompatibility of the IR783/BC NPs and its components in dark (FIG. 8A).

Subsequently, the anti-proliferation effect was evaluated with HCT116 cells by MTT assay (FIG. 3B). Upon 530 nm light irradiation, the cell viability decreased gradually to nearly 0% by increasing IR783/BC NPs concentration, with a IC₅₀ value of 6.62 μM on BC prodrug basis. The nanoparticles exhibited better therapeutic effect than the free BC prodrug under light irradiation (IC₅₀ value: 9.24 μM), which can be explained by the nanoparticle have better dispersion ability in the medium compared to the free prodrug. Besides, the cytotoxicity of the free anticancer drug chlorambucil was demonstrated to be relatively low at the experimental conditions (FIG. 8B). It should be noted that chlorambucil was observed to rapidly hydrolyze in the aqueous solution, resulting in the loss of the cytotoxicity [17, 39]. Thus, the prodrug strategy effectively enhanced the therapeutics efficacy of chlorambucil. Here, the cytotoxicity studies verified that the IR783/BC NPs exhibited enhanced therapeutics effect against HCT116 cancer cells upon light irradiation.

To further investigate the therapeutic effect of the nanoparticles, live-dead staining analysis was conducted by Calcein AM/PI co-staining. As shown in FIG. 3C, the dead cells presenting red fluorescence were observed both in the irradiated BC-treated group and IR783/BC NPs-treated group while other groups without light irradiation or BC molecule did not cause significant cell death, which coincided well with the results of the cytotoxicity study. Additionally, apoptosis study of HCT116 cells treated with free chlorambucil or IR783/BC NPs was conducted by Annexin-V FITC/PI assay to investigate the anticancer activity (FIGS. 9A-9I). Cell apoptosis rate was significantly elevated after treating with IR783/BC NPs and light irradiation. At the concentration of 20 μM, the nanoparticles plus light irradiation induced 77.13% of cell apoptosis, which was mainly dominated by the late apoptosis (70.45%). In comparison, free chlorambucil at the same concentration only caused about 5% apoptosis no matter whether the light irradiation was applied or not, since the free chlorambucil cannot enter the cells as efficient as the NPs. In all, these results confirmed the effective cytotoxicity and apoptosis-inducing effect of IR783/BC NPs with light irradiation.

### Example 4

### Light-Triggered ¹O₂ Generation From IR783/BC NPS

Interestingly, compared with free chlorambucil, the BC prodrug irradiated with 530 nm light showed higher cytotoxicity. One of the reasons should be that the iodide containing BODIPY group of BC prodrug exhibits high inter-system crossing efficiency and enhances singlet oxygen production ability upon light irradiation [40, 41]. For confirmation, ¹O₂ generation of IR783/BC NPs was determined by the Singlet Oxygen Sensor Green® (SOSG) Assay. SOSG is a commonly used indicator of ¹O₂ that can unequivocally indicate the ¹O₂ generation via tunable photoinduced

electron transfer (PET) mechanism [42]. As shown in FIG. 3D, in the solution containing SOSG and IR783/BC NPs with light irradiation (530 nm, 50 mW/cm², 0-1 min), the fluorescence of SOSG significantly increased in the range of 500-600 nm, indicating the ¹O₂ generation from IR783/BC NPs with light irradiation. In comparison, we also observed that the generation of ¹O₂ from free BC prodrug was similar to that of IR783/BC NPs at the equivalent concentration of BC, while the free IR783 dye generated ¹O₂ at a much lower efficiency (FIG. 3E). Therefore, the photodynamic effect of IR783/BC NPs also contributed to the cytotoxicity of the BC prodrug upon light irradiation.

The intracellular singlet oxygen generation by IR783/BC NPs was investigated in HCT116 cells by DCFH-DA (2,7-dichlorodihydrofluorescein diacetate). DCFH-DA is non-fluorescent in nature whereas forming dichloro-fluorescein with strong fluorescence in the exist of ¹O₂. As shown in FIG. 3F, HCT116 cells co-incubated with both IR783/BC NPs and DCFH-DA exhibited little green fluorescence, indicating negligible ¹O₂ generation in dark. After 10-min irradiation at 50 mW/cm², strong intracellular green fluorescence was observed by confocal imaging, which demonstrated the intracellular ¹O₂ generation by IR783/BC NPs upon light irradiation.

### Example 5

### In Vivo Biodistribution and In-Situ Monitoring

As aforementioned, the sulfate groups of the IR783 dye may attribute to the tumor-targeting performance of IR783/BC NPs by CAV-1-mediated transcytosis. In addition, fluorescence imaging based on the emission changes of IR783 upon light irradiation could be expected to provide the possibility for in vivo monitoring of drug delivery (FIG. 4A). The fluorescence imaging of HCT116-tumor-bearing mice was recorded at 1, 6, 16, 20 and 24 h after free IR783 and IR783/BC NPs injected into the mice intravenously (FIG. 4B). The fluorescence of free IR783 fleetly eliminated after the systemic administration, due to the fast removal of the free dye from the blood circulation. In contrast, IR783/BC NPs showed a longer retention post-injection. The intensity of the NIR fluorescence in the tumor region gradually increased within 24 h after the injection of IR783/BC NPs, indicating that the nanoparticles possessed enhanced accumulation in the HCT116 tumors compared to the free dye, which can be explained by the combination of both the EPR effect and the active recognition of IR783/BC NPs towards HCT116 tumor cells. In addition, the tumors and main organs were separated for ex vivo fluorescence imaging 24 h after administration (FIG. 4C and FIG. 10). Compared to free IR783, the nanoparticles exhibited much better tumor retention capability. Besides, obviously preferential accumulation of the nanoparticles was observed in the tumors and liver. It should be noted that we can selectively activate the nanoparticles in the tumors by light while those nanoparticles in the liver will not be activated, which may reduce the side effects.

The in-situ monitoring of photoinduced nanoparticle dissociation was examined in HCT116 tumor-bearing mice. As shown in FIG. 4D, it was observed that all the three mice treated with IR783/BC NPs showed strong fluorescence at the tumor sites 24 h post-injection, displaying the accumulation of the nanoparticles. The 530 nm light irradiation (100 mW/cm²) was applied topically on the tumors with various durations from 0 to 300 s. The fluorescence images were recorded right after the irradiation was finished, with a cumulative irradiation duration of 0, 30, 60, 120, 300 s, separately. The results revealed that the fluorescence intensity in the tumors gradually decreased with the increase of the irradiation duration, which was consistent with in vitro photoresponsive properties of IR783/BC NPs (FIG. 4E). Therefore, the in vivo fluorescence imaging enabled in-situ monitoring of the drug delivery, including nanoparticles accumulation and light-triggered nanoparticles dissociation. The real-time monitoring of such phototherapy may provide important guidance to indicate the in vivo distribution and the real-time decomposition of the nanomedicine, which can be utilized to reduce the overdose of drug or light.

### Example 6

### Antitumor Efficacy of IR783/NPs

Encouraged by the in vitro cytotoxicity study and in vivo biodistribution study, we subsequently performed in vivo efficacy study to illustrate the advantages of the photoresponsive IR783/BC NPs. BALB/c-nude mice with HCT116 xenografts with tumor size at around 200 mm³ were randomly divided into 6 groups (a. PBS; b. PBS+hv; c. IR783+hv; d. free Cb; e. IR783/BC NPs; f. IR783/BC NPs+hv) and the corresponding formulations were intravenously injected. At 24 h post-injection, 530 nm light irradiation (100 mW/cm², 10 min) was applied topically onto the tumors (FIG. 5A). The treatments were repeated on Day 0, 4, 8 and 12. As shown in FIGS. 5B-5D, treatment with IR783/BC NPs plus light irradiation significantly suppressed the tumor growth while other groups only displayed limited therapeutics effects. It should be noted that the anti-tumor efficacy of free chlorambucil was undermined in vivo, which may be due to its hydrolysis

and fast clearance in blood [39]. The nanoparticles without light irradiation also showed poor efficacy, indicating the therapeutics effect of IR783/BC NPs would only be activated under light irradiation. Additionally, no remarkable body weight loss was observed during the treatments with these formulations, suggesting good biocompatibility of the treatments (FIG. 5G).

After 14-day monitoring, the mice were sacrificed, and the tumors were resected for ex vivo characterizations. The tumor size and weight of the group treated with IR783/BC NPs plus light irradiation were significantly lower than other groups, which further supported the above results (FIGS. 5E and 5F). Moreover, the cell proliferation and apoptosis in tumors were analyzed by hematoxylin and eosin (H&E) staining and immunohistochemistry staining, respectively. The results shown in FIG. 5H revealed that IR783/BC NPs with light irradiation effectively inhibited proliferation and induced apoptosis in tumor tissues. As expected, immunohistochemistry staining of CAV-1 also confirmed its high expression in all the tumors. Moreover, based on the H&E staining results (FIG. 11), the main organs exhibited no apparent necrosis or histological change after the treatments, suggesting minimal systemic toxicity. All these in vivo data suggested that the IR783/BC NPs could serve as a promising therapeutic agent against tumors with light-controllable activity, resulting in enhanced anti-tumor efficacy and safety.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Rautio J, Kumpulainen H, Heimbach T, Oliyai R, Oh D, Järvinen T, et al. Prodrugs: design and clinical applications. Nature reviews Drug discovery. 2008; 7:255-70.
2. Zhong Y, Shao L, Li Y. Cathepsin B-cleavable doxorubicin prodrugs for targeted cancer therapy. Int J Oncol. 2013; 42:373-83.
3. Xu Y, Mu J, Xu Z, Zhong H, Chen Z, Ni Q, et al. Modular acid-activatable acetone-based ketal-linked nanomedicine by dexamethasone prodrugs for enhanced anti-rheumatoid arthritis with low side effects. Nano Lett. 2020; 20:2558-68.
4. Gao C, Tang F, Gong G, Zhang J, Hoi MP, Lee SM, et al. pH-responsive prodrug nanoparticles based on a sodium alginate derivative for selective co-release of doxorubicin and curcumin into tumor cells. Nanoscale. 2017; 9:12533-42.
5. Kalva N, Uthaman S, Augustine R, Jeon SH, Huh KM, Park IK, et al. Photo—and pH—Responsive Polycarbonate Block Copolymer Prodrug Nanomicelles for Controlled Release of Doxorubicin. Macromol Biosci. 2020; 20:2000118.
6. Hu Q, Katti PS, Gu Z. Enzyme-responsive nanomaterials for controlled drug delivery. Nanoscale. 2014; 6:12273-86.
7. Yu N, Liu T, Zhang X, Gong N, Ji T, Chen J, et al. Dually enzyme-and acid-triggered self-immolative ketal glycoside nanoparticles for effective cancer prodrug monotherapy. Nano Lett. 2020; 20:5465-72.
8. Sirsi SR, Borden MA. State-of-the-art materials for ultrasound-triggered drug delivery. Adv Drug Del Rev. 2014; 72:3-14.
9. May JP, Li S-D. Hyperthermia-induced drug targeting. Expert opinion on drug delivery. 2013; 10:511-27.
10. McDaniel JR, Bhattacharyya J, Vargo KB, Hassouneh W, Hammer DA, Chilkoti A. Self—assembly of thermally responsive nanoparticles of a genetically encoded peptide polymer by drug conjugation. Angew Chem Int Ed. 2013; 52:1683-7.
11. Rwei AY, Wang W, Kohane DS. Photoresponsive nanoparticles for drug delivery. Nano today. 2015; 10:451-67.
12. Li Y, Zhang Y, Wang W. Phototriggered targeting of nanocarriers for drug delivery. Nano Research. 2018; 11:5424-38.
13. Brieke C, Rohrbach F, Gottschalk A, Mayer G, Heckel A. Light-controlled tools. Angew Chem Int Ed. 2012; 51:8446-76.
14. Liu M, Meng J, Bao W, Liu S, Wei W, Ma G, et al. Single-Chromophore-Based Therapeutic Agent Enables Green-Light-Triggered Chemotherapy and Simultaneous Photodynamic Therapy to Cancer Cells. ACS Applied Bio Materials. 2019; 2:3068-76.
15. Chaudhuri A, Venkatesh Y, Das J, Behara KK, Mandal S, Maiti TK, et al. Squaric acid-coumarin-chlorambucil: photoresponsive single-component fluorescent organic nanoconjugates for self-monitored therapeutics. ACS Applied Nano Materials. 2018; 1:63 12-9.
16. Liu L, Qiu W, Li B, Zhang C, Sun L, Wan S, et al. A Red Light Activatable Multifunctional Prodrug for Image-Guided Photodynamic Therapy and Cascaded Chemotherapy. Adv Funct Mater. 2016; 26:6257-69.
17. Lv W, Long K, Yang Y, Chen S, Zhan C, Wang W. A Red Light—Triggered Drug Release System Based on One—Photon Upconversion—Like Photolysis. Advanced Healthcare Materials. 2020; 9:2001118.
18. Mueller CE. Prodrug approaches for enhancing the bioavailability of drugs with low solubility. Chem Biodivers. 2009; 6:2071-83.
19. Stella VJ, Nti-Addae KW. Prodrug strategies to overcome poor water solubility. Adv Drug Del Rev. 2007; 59:677-94.
20. Kolega J. Phototoxicity and photoinactivation of blebbistatin in UV and visible light. Biochem Biophys Res Commun. 2004; 320:1020-5.
21. Weissleder R. A clearer vision for in vivo imaging. Nat Biotechnol. 2001; 19:316-7.
22. Liu Q, Wang W, Zhan C, Yang T, Kohane DS. Enhanced precision of nanoparticle phototargeting in vivo at a safe irradiance. Nano Lett. 2016; 16:4516-20.
23. Long K, Yang Y, Lv W, Jiang K, Li Y, Lo ACY, et al. Green Light-Triggered Intraocular Drug Release for Intravenous Chemotherapy of Retinoblastoma. Advanced Science. 2021:2101754.
24. Xiao J, Cheng L, Fang T, Zhang Y, Zhou J, Cheng R, et al. Nanoparticle—Embedded Electrospun Fiber—Covered Stent to Assist Intraluminal Photodynamic Treatment of Oesophageal Cancer. Small. 2019; 15:1904979.
25. Yamagishi K, Kirino I, Takahashi I, Amano H, Takeoka S, Morimoto Y, et al. Tissue-adhesive wirelessly powered optoelectronic device for metronomic photodynamic cancer therapy. Nature biomedical engineering. 2019; 3:27-36.
26. Luo S, Zhang E, Su Y, Cheng T, Shi C. A review of NIR dyes in cancer targeting and imaging. Biomaterials. 2011; 32:7127-38.
27. Shamay Y, Shah J, Isik M, Mizrachi A, Leibold J, Tschaharganeh DF, et al. Qua ntitative self-assembly prediction yields targeted nanomedicines. Nat Mater. 2018; 17:361-8.
28. Voigt J, Christensen J, Shastri VP. Differential uptake of nanoparticles by endothelial cells through polyelectrolytes with affinity for caveolae. Proceedings of the National Academy of Sciences. 2014; 111:2942-7.
29. Nani RR, Gorka AP, Nagaya T, Kobayashi H, Schnermann MJ. Near-IR Light-Mediated Cleavage of Antibody-Drug Conjugates Using Cyanine Photocages. Angew Chem Int Ed Engl. 2015; 54:13635-8.
30. Gorka AP, Yamamoto T, Zhu J, Schnermann MJ. Cyanine Photocages Enable Spatial Control of Inducible Cre-Mediated Recombination. ChemBioChem. 2018; 19:1239-43.
31. Nani RR, Gorka AP, Nagaya T, Yamamoto T, Ivanic J, Kobayashi H, et al. In Vivo Activation of Duocarmycin-Antibody Conjugates by Near-Infrared Light. ACS Cent Sci. 2017; 3:329-37.
32. Lv W, Li Y, Li F, Lan X, Zhang Y, Du L, et al. Upconversion-like Photolysis of BODIPY-Based Prodrugs via a One-Photon Process. J Am Chem Soc. 2019; 141:17482-6.
33. Meng X, Li W, Sun Z, Zhang J, Zhou L, Deng G, et al. Tumor-targeted small molecule for dual-modal imaging-guided phototherapy upon near-infrared excitation. Journal of Materials Chemistry B. 2017; 5:9405-11.
34. Uhrich KE, Cannizzaro SM, Langer RS, Shakesheff KM. Polymeric systems for controlled drug release. Chem Rev. 1999, 99:3181-98.
35. Luo S, Tan X, Qi Q, Guo Q, Ran X, Zhang L, et al. A multifunctional heptamethine near-infrared dye for cancer theranosis. Biomaterials. 2013; 34:2244-51.
36. Zhang Y, Sha R, Zhang L, Zhang W, Jin P, Xu W, et al. Harnessing copper-palladium alloy tetrapod nanoparticle-induced pro-survival autophagy for optimized photothermal therapy of drug-resistant cancer. Nat Commun. 2018; 9:4236.
37. Li Z, Wang N, Huang C, Bao Y, Jiang Y, Zhu G. Downregulation of caveolin-1 increases the sensitivity of drug-resistant colorectal cancer HCT116 cells to 5-fluorouracil. Oncol Lett. 2017; 13:483-7.
38. Basu Roy UK, Henkhaus RS, Loupakis F, Cremolini C, Gerner EW, Ignatenko NA. Caveolin-1 is a novel regulator of K-RAS-dependent migration in colon carcinogenesis. Int J Cancer. 2013; 133:43-57.
39. Negreira N, Mastroianni N, de Alda ML, Barceló D. Multianalyte determination of 24 cytostatics and metabolites by liquid chromatography-electrospray-tandem mass spectrometry and study of their stability and optimum storage conditions in aqueous solution. Talanta. 2013; 116:290-9.
40. Miao X, Hu W, He T, Tao H, Wang Q, Chen R, et al. Deciphering the intersystem crossing in near-infrared BODIPY photosensitizers for highly efficient photodynamic therapy. Chemical science. 2019; 10:3096-102.
41. Zou J, Yin Z, Ding K, Tang Q, Li J, Si W, et al. BODIPY derivatives for photodynamic therapy: influence of configuration versus heavy atom effect. ACS applied materials & interfaces. 2017; 9:32475-81.
42. Kim S, Fujitsuka M, Majima T. Photochemistry of singlet oxygen sensor green. The journal of physical chemistry B. 2013; 117:13985-92.

1. A composition comprising a boron-dipyrromethene-derived compound and a dye.

2. The composition of claim 1, wherein the boron-dipyrromethene-derived compound is one or more selected from boron-dipyrromethene-chlorambucil (BC) according to formula (Ia), boron-dipyrromethene-naproxen (BN) according to formula (Ib), boron-dipyrromethene-benzyloxycinnamic acid (BBA) according to formula (Ic), and boron-dipyrromethene-dopamine (BD) according to formula (Id):

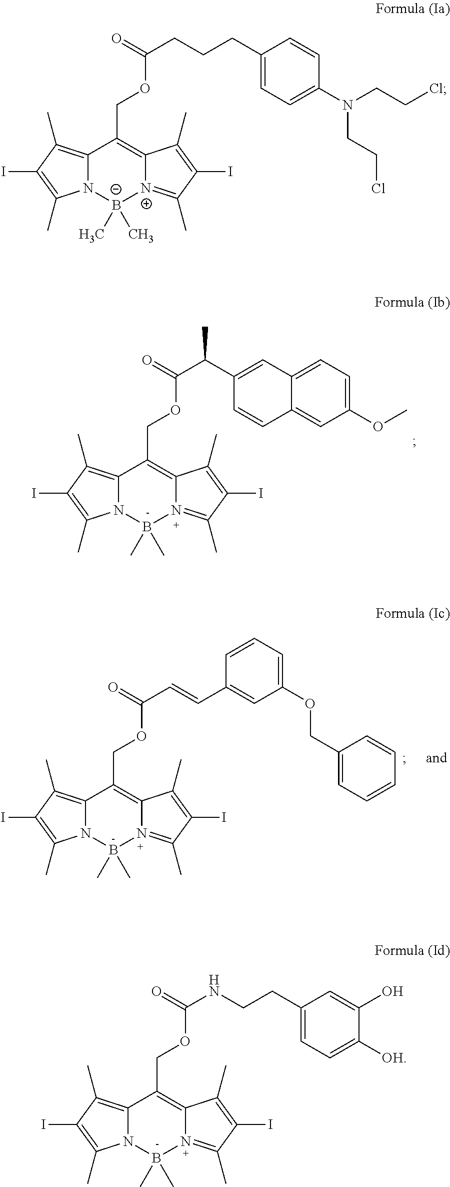 [SMILES: CC1=C(I)C(C)=[N+]2C1=C(COC(=O)CCCc1ccc(N(CCCl)CCCl)cc1)c1c(C)c(I)c(C)n1[B-]2(C)C ; COc1ccc2cc([C@H](C)C(=O)OCC3=C4C(C)=C(I)C(C)=[N+]4[B-](C)(C)n4c(C)c(I)c(C)c43)ccc2c1 ; CC1=C(I)C(C)=[N+]2C1=C(COC(=O)/C=C/c1cccc(OCc3ccccc3)c1)c1c(C)c(I)c(C)n1[B-]2(C)C ; CC1=C(I)C(C)=[N+]2C1=C(COC(=O)NCCc1ccc(O)c(O)c1)c1c(C)c(I)c(C)n1[B-]2(C)C]

3. The composition of claim 1, wherein the dye is a near-infrared dye.

4. The composition of claim 1, wherein the dye is a near-infrared cyanine dye.

5. The composition of claim 1, wherein the dye contains sulfate groups.

6. The composition of claim 1, wherein the dye is IR783, IR820, or ICG.

7. The composition of claim 1, wherein the composition is a nanoparticle.

8. The composition of claim 1, wherein the composition is about 0.1% to about 99.9% boron-dipyrromethene-derived compound.

9. The composition of claim 1, wherein the composition is about 0.1% to about 10% dye.

10. A method of in-situ fluorescence imaging of light-triggered dissociation of the composition of claim 1, said method comprising:
a) administering the composition of claim 1 to a subject;
b) irradiating the composition of claim 1, wherein the irradiation cleaves a photocleavable group of the boron-dipyrromethene-derived compound and dissociates the composition of claim 1; and
c) measuring the fluorescence of the dye of the composition of claim 1.

11. The method of claim 10, wherein the irradiation is non-ionizing radiation at a wavelength of about 100 nm to about 1000 nm, about 200 nm to about 700 nm, about 350 nm to about 550 nm, or about 530 nm.

12. The method of claim 10, wherein the irradiation occurs for about 1 second to about 10 minutes.

13. The method of claim 10, wherein the irradiance of the irradiation is about 1 mW/cm² to about 1000 mW/cm², about 5 mW/cm² to about 500 mW/cm², about 10 mW/cm² to about 250 mW/cm², about 15 mW/cm² to about 150 mW/cm², about 25 mW/cm² to about 125 mW/cm², about 50 mW/cm² to about 100 mW/cm², or about 100 mW/cm².

14. A method of inhibiting tumor growth, said method comprising:
a) contacting the composition of claim 1 with a tumor cell; and
b) irradiating the composition of claim 1, wherein the irradiation cleaves a photocleavable group of the boron-dipyrromethene-derived compound and releases singlet oxygen.

15. The method of claim 14, wherein the irradiation is non-ionizing radiation at a wavelength of about 100 nm to about 1000 nm, about 200 nm to about 700 nm, about 350 nm to about 550 nm, or about 530 nm.

16. The method of claim 14, wherein the irradiation occurs for about 1 second to about 10 minutes.

17. The method of claim 14, wherein the irradiance of the irradiation is about 1 mW/cm² to about 1000 mW/cm², about 5 mW/cm² to about 500 mW/cm², about 10 mW/cm² to about 250 mW/cm², about 15 mW/cm² to about 150 mW/cm², about 25 mW/cm² to about 125 mW/cm², about 50 mW/cm² to about 100 mW/cm², or about 100 mW/cm².

18. A method of transcytosis, said method comprising:
a) contacting the composition of claim 5 with a cell; and
b) irradiating the composition of claim 5, wherein the irradiation cleaves a photocleavable group of the boron-dipyrromethene-derived compound and sulfate groups of the dye increase the accumulation of the composition of claim 5 in the cell by CAV-1-mediated transcytosis.

19. The method of claim 18, wherein the irradiation is non-ionizing radiation at a wavelength of about 100 nm to about 1000 nm, about 200 nm to about 700 nm, about 350 nm to about 550 nm, or about 530 nm.

20. The method of claim 18, wherein the irradiation occurs for about 1 second to about 10 minutes.

21. The method of claim 18, wherein the irradiance of the irradiation is about 1 mW/cm² to about 1000 mW/cm², about 5 mW/cm² to about 500 mW/cm², about 10 mW/cm² to about 250 mW/cm², about 15 mW/cm² to about 150 mW/cm², about 25 mW/cm² to about 125 mW/cm², about 50 mW/cm² to about 100 mW/cm², or about 100 mW/cm².
